(19) United States
(12) Patent Application Publication
(10) Pub. No.: US 2008/0207718 A1
(43) Pub. Date: Aug. 28, 2008

(54) Use of Fused Imidazole Derivatives to Mediate Ccr3 Related Conditions

(76) Inventors: Rohan Beckwith, Cambridge, MA (US); Kate Hoegenauer, Wien (AT); Jeremy Lee Jenkins, Watertown, MA (US); Philipp Lehr, Wien (AT); Thomas Ullrich, Wien (AT); Klaus Weigand, Wien (AT)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/065,239

(22) PCT Filed: Aug. 31, 2006
(86) PCT No.: PCT/EP2006/008516

§ 371 Date: Feb. 28, 2008

(30) Foreign Application Priority Data
Sep. 2, 2005 (GB) 0517966.8

(51) Int. Cl. A61K 31/4164; C07D 487/04; A61P 29/00; A61P 11/00

(52) U.S. Cl. 514/393; 548/302.7

(57) ABSTRACT

The use of compound of formula I in the preparation of a medicament, e.g. for the treatment of condition mediated by CCR3.

The present invention relates to organic compounds, e.g. the use of compounds of given formula in the preparation of a medicament.

In one aspect the present invention provides a compound of formula

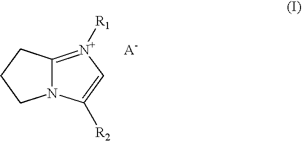

wherein
R₁ is—unsubstituted (C₁₋₆)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms;
   (C₆₋₁₈)aryl, (C₆₋₁₈)aryl(C₁₋₆)alkyl, (C₆₋₁₈)aryl-carbonyl(C₁₋₄)alkyl, heterocyclyl, heterocyclyl(C₁₋₆)alkyl, heterocyclyl-carbonyl(C₁₋₆)alkyl, wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₁₋₆)alkyl, (C₁₋₄)alkoxy, (C₃₋₈)cycloalkyl, nitro, halo(C₁₋₄)alkyl or sulfanyl(C₁₋₄)alkyl;
   aminocarbonyl(C₁₋₆)alkyl in unsubstituted form or one or morefold substituted by
   (C₁₋₆)alkyl;
   (C₃₋₈)cycloalkyl,
   halo(C₁₋₄)alkyl,
   halogen,
   unsubstituted (C₆₋₁₈)aryl,
   (C₆₋₁₈)aryl substituted by (C₁₋₆)alkyl, (C₁₋₄)alkoxy, (C₃₋₈)cycloalkyl, nitro,
   halo(C₁₋₄)alkyl, halogen,
   (C₆₋₁₈)aryl annelated with (C₆₋₁₈)aryl or heterocyclyl, wherein heterocyclyl has 5 or 6 ring members and 1 to 4 heteroatoms selected from N, O, S,
   unsubstituted heterocyclyl, wherein heterocyclyl has 5 or 6 ring members and 1 to 4 heteroatoms selected from N, O, S,
   heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₁₋₄)alkyl, halogen,
   heterocyclyl annelated with (C₆₋₁₈)aryl or heterocyclyl, wherein heterocyclyl has 5 or 6 ring members and 1 to 4 heteroatoms selected from N, O, S,
   R₂ is—unsubstituted (C₆₋₁₈)aryl or (C₆₋₁₈)aryl substituted by (C₁₋₄)alkyl, (C₁₋₄)alkoxy, (C₁₋₄)haloalkyl or halogen; and
   A⁻ is a pharmaceutically acceptable anion,
   in the preparation of a medicament.

In one aspect the present invention provides a compound of formula (I) wherein
R₁ is—unsubstituted (C₁₋₆)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms;
   (C₆₋₁₈)aryl, (C₆₋₁₈)aryl-carbonyl(C₁₋₄)alkyl, heterocyclyl, heterocyclyl(C₁₋₆)alkyl, heterocyclyl-carbonyl(C₁₋₆)alkyl, wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₁₋₆)alkyl, (C₁₋₄)alkoxy, (C₃₋₈)cycloalkyl, nitro, halo(C₁₋₄)alkyl, sulfanyl(C₁₋₄)alkyl or halogen;
   aminocarbonyl(C₁₋₆)alkyl in unsubstituted form or one or morefold substituted by
   (C₁₋₆)alkyl;
   (C₃₋₈)cycloalkyl,
   halo(C₁₋₄)alkyl,
   halogen,
   unsubstituted (C₆₋₁₈)aryl,
   (C₆₋₁₈)aryl substituted by (C₁₋₆)alkyl, (C₁₋₄)alkoxy, (C₃₋₈)cycloalkyl, nitro,
   halo(C₁₋₄)alkyl, halogen,
   (C₆₋₁₈)aryl annelated with (C₆₋₁₈)aryl or heterocyclyl, wherein heterocyclyl has 5 or 6 ring members and 1 to 4 heteroatoms selected from N, O, S,
   unsubstituted heterocyclyl, wherein heterocyclyl has 5 or 6 ring members and 1 to 4 heteroatoms selected from N, O, S,
   heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₁₋₄)alkyl, halogen,
   heterocyclyl annelated with (C₆₋₁₈)aryl or heterocyclyl, wherein heterocyclyl has 5 or 6 ring members and 1 to 4 heteroatoms selected from N, O, S,
R₂ and A⁻ are as defined above,
   in the preparation of a medicament.

In another aspect the present invention provides a compound of formula (I), wherein (C₆₋₁₈)aryl is phenyl, optionally annelated with phenyl or heterocyclyl, wherein heterocyclyl has 5 or 6 ring members and 1 to 4 heteroatoms selected from N, O, S.

In another aspect the present invention provides a compound of formula (I), wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl, unsubstituted phenyl or phenyl one or morefold substituted by (C₁₋₄)alkyl, (C₁₋₂)alkoxy, halogen, nitro; unsubstituted phenyl(C₁₋₄)alkyl or phenyl(C₁₋₄)alkyl one or morefold substituted by (C₁₋₄)alkyl, (C₁₋₂)alkoxy, nitro;
   unsubstituted phenyl-carbonyl(C₁₋₂)alkyl or phenyl-carbonyl(C₁₋₂)alkyl one or morefold substituted by (C₁₋₄)alkyl, (C₁₋₂)alkoxy, halogen, nitro, (C₃₋₈)cycloalkyl, (C₁₋₄)alkyl-sulfanyl, heterocyclyl-(C₁₋₄)alkyl, wherein heterocyclyl has 5 or 6 ring members and 1 to 2 heteroatoms selected from N, S, optionally annelated with phenyl;
   heterocyclyl-carbonyl(C₁₋₄)alkyl, wherein heterocyclyl has 5 or 6 ring members and 1 to 2 heteroatoms selected from N, S, optionally annelated with phenyl;
   unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl.
R₂ is unsubstituted phenyl or phenyl substituted by (C₁₋₄)alkyl, (C₁₋₄)alkoxy or halogen,
A⁻ is as defined above.

In another aspect the present invention provides a compound of formula (I), wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl, unsubstituted phenyl or phenyl one or morefold substituted by (C₁₋₄)alkyl, (C₁₋₂)alkoxy, halogen, nitro; unsubstituted phenyl(C₁₋₄)alkyl,
   unsubstituted phenyl-carbonyl(C₁₋₂)alkyl or phenyl-carbonyl(C₁₋₂)alkyl one or morefold substituted by (C₁₋₄)alkyl, (C₁₋₂)alkoxy, halogen, nitro, (C₃₋₈)cycloalkyl, (C₁₋₄)alkyl-sulfanyl, heterocyclyl-(C₁₋₄)alkyl, wherein heterocyclyl has 5 or 6 ring members and 1 to 2 heteroatoms selected from N, S, optionally annelated with phenyl; heterocyclyl-carbonyl(C₁₋₄)alkyl, wherein heterocyclyl has 5 or 6 ring members and 1 to 2 heteroatoms selected from N, S, optionally annelated with phenyl;
   unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl.
R₂ is unsubstituted phenyl or phenyl substituted by (C₁₋₄)alkyl, (C₁₋₄)alkoxy or halogen,
A⁻ is as defined above.

If not otherwise defined herein
Alkyl includes (C₁₋₈)alkyl, e.g. (C₁₋₆)alkyl, such as e.g. (C₁₋₄)alkyl;
Alkoxy includes (C₁₋₈)alkoxy, e.g. (C₁₋₆)alkoxy, such as e.g. (C₁₋₄)alkoxy;
(C₃₋₈)cycloalkyl includes e.g. (C₃₋₆)cycloalkyl; such as e.g. cyclohexyl;
Aryl includes (C₆₋₁₈)aryl, e.g. phenyl; optionally anellated with (C₆₋₁₈)aryl, e.g. phenyl or with heterocyclyl, e.g. heterocyclyl having 6 ring members and 2 O as heteroatoms, such as e.g. dioxine;
Heterocyclyl includes a 5 or 6 membered ring having 1 to 4 heteroatoms selected from S, O and N; e.g. N, S; such as e.g. thiophene or thiazole;
optionally anellated with a further ring (system), e.g. anellated with one or more (C₆₋₁₈)aryl, e.g. phenyl, or anellated with heterocyclyl;
Halogen includes fluoro, chloro, bromo;
Haloalkyl includes halo(C₁₋₄)alkyl, wherein halo is one or more halogen, preferably trifluoromethyl;

Any group may be unsubstituted or substituted, e.g. substituted by groups as conventional in organic chemistry, e.g. including groups selected from halogen, haloalkyl, alkylcarbonyloxy, alkoxy, hydroxy, amino, alkylcarbonylamino, aminoalkylcarbonylamino, hydroxyalkylamino, aminoalkylamino, alkylamino, dialkylamino, heterocyclyl having 5 or 6 ring members and 1 to 4 heteroatoms selected from N, O, S; (C₁₋₄)alkylheterocyclyl, wherein heterocyclyl having 5 or 6 ring members and 1 to 4 heteroatoms selected from N, O, S; hydroxy(C₁₋₄)alkylheterocyclyl, wherein heterocyclyl having 5 or 6 ring members and 1 to 4 heteroatoms selected from N, O, S; carboxyl, (C₁₋₄)alkylcarbonyloxy, amino(C₁₋₄)-alkylcarbonyloxy.

In a compound of formula I each single defined substitutent may be a preferred substituent, e.g. independently of each other substitutent defined.

In another aspect the present invention provides a compound of formula (I) with the proviso that a compound of example 1 to 378 is excluded.

Compounds used by or provided by the present invention are hereinafter designated as “compound(s) of (according to) the present invention”. A compound of the present invention includes a compound in the form of a salt, in the form of a solvate and in the form of a salt and a solvate.

The compound of the present invention is present in the form of a salt.

Such salts include preferably pharmaceutically acceptable salts, although pharmaceutically unacceptable salts are included, e.g. for preparation/isolation/purification purposes. The anion A⁻ is a pharmaceutically acceptable anion, such as from an inorganic acid, e.g. hydrohalic acids such as hydrofluoric acid, hydrochloric-acid, hydrobromic acid or hydroiodic acid, nitric acid, sulfuric acid, phosphoric acid; and from an organic acid, for example aliphatic monocarboxylic acids such as formic acid, acetic acid, trifluoroacetic acid, propionic acid and butyric acid, aliphatic hydroxyl acids such as lactic acid, citric acid, tartaric acid or malic acid; dicarboxylic acid such as maleic acid or succinic acid, aromatic carboxylic acids such as benzoic acid, p-chlorobenzoic acid, diphenylacetic acid or triphenylacetic acid, aromatic hydroxyl acids such as o-hydroxybenzoic acid, p-hydroxybenzoic acid, 1-hydroxynaphthalene-2-carboxylic acid or 3-hydroxynaphtalene-2-carboxylic acid, and sulfonic acids such as methanesulfonic acid or benzenesulfonic acid.

Preferably A⁻ is halogen, e.g. bromide and chloride, most preferred chloride.

A compound of the present invention in the form of a salt and in the form of a solvate may be converted into a corresponding compound in the form of a salt in non-solvated form; and vice versa.

A compound of the present invention may exist in the form of pure isomers or mixtures thereof; e.g. optical isomers, diastereoisomers, cis/trans isomers. A compound of the present invention may e.g. contain asymmetric carbon atoms and may thus exist in the form of enantiomers or diastereoisomers and mixtures thereof, e.g. racemates. Any asymmetric carbon atom may be present in the (R)-, (S)- or (R,S)-configuration, preferably in the (R)- or (S)-configuration. Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

The compounds of the present invention, e.g. including a compound of formula I, exhibit pharmacological activity and are therefore useful as pharmaceuticals. E.g., the compounds of formula I are useful in the preparation of a medicament for the treatment of a condition mediated by CCR3.

In another aspect the present invention provides a compound of formula (I), wherein
R₁ is—unsubstituted (C₁₋₆)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms;
   (C₆₋₁₈)aryl, (C₆₋₁₈)aryl(C₁₋₆)alkyl, (C₆₋₁₈)aryl-carbonyl(CIA)alkyl, heterocyclyl, heterocyclyl(C₁₋₆)alkyl, heterocyclyl-carbonyl(C₁₋₆)alkyl, wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₁₋₆)alkyl, (C₁₋₄)alkoxy, (C₃₋₈)cycloalkyl, nitro, halo(C₁₋₄)alkyl, halogen or sulfanyl(C₁₋₄)alkyl;
   aminocarbonyl(C₁₋₆)alkyl in unsubstituted form or one or morefold substituted by
   (C₁₋₆)alkyl;
   (C₃₋₈)cycloalkyl,
   halo(C₁₋₄)alkyl,
   halogen,
   unsubstituted (C₆₋₁₈)aryl,
   (C₆₋₁₈)aryl substituted by (C₁₋₆)alkyl, (C₁₋₄)alkoxy, (C₃₋₈)cycloalkyl, nitro, halo(C₁₋₄)alkyl, halogen,
   (C₆₋₁₈)aryl annelated with (C₆₋₁₈)aryl or heterocyclyl, wherein heterocyclyl has 5 or 6 ring members and 1 to 4 heteroatoms selected from N, O, S,
   unsubstituted heterocyclyl, wherein heterocyclyl has 5 or 6 ring members and 1 to 4 heteroatoms selected from N, O, S,
   heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₁₋₄)alkyl, halogen,
   heterocyclyl annelated with (C₆₋₁₈)aryl or heterocyclyl, wherein heterocyclyl has 5 or 6 ring members and 1 to 4 heteroatoms selected from N, O, S,
R₂ is—unsubstituted (C₆₋₁₈)aryl or (C₆₋₁₈)aryl substituted by (C₁₋₄)alkyl, (C₁₋₄)alkoxy, (C₁₋₄)haloalkyl or halogen;
A⁻ is a pharmaceutically acceptable anion,
   in the preparation of a medicament for the treatment of a condition mediated by CCR3.

In another aspect the present invention provides a compound of formula (I), wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl,
   unsubstituted phenyl or phenyl one or morefold substituted by (C₁₋₄)alkyl, (C₁₋₂)alkoxy, halogen, nitro;
   unsubstituted phenyl(C₁₋₄)alkyl or phenyl(C₁₋₄)alkyl one or morefold substituted by (C₁₋₄)alkyl, (C₁₋₂)alkoxy, halogen, nitro;
   unsubstituted phenyl-carbonyl(C₁₋₂)alkyl or phenyl-carbonyl(C₁₋₂)alkyl one or morefold substituted by (C₁₋₄)alkyl, (C₁₋₂)alkoxy, halogen, nitro, (C₃₋₈)cycloalkyl, (C₁₋₄)alkyl-sulfanyl, heterocyclyl-(C₁₋₄)alkyl, wherein heterocyclyl has 5 or 6 ring members and 1 to 2 heteroatoms selected from N, S, optionally annelated with phenyl;
   heterocyclyl-carbonyl(C₁₋₄)alkyl, wherein heterocyclyl has 5 or 6 ring members and 1 to 2 heteroatoms selected from N, S, optionally annelated with phenyl;
   unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl;
R₂ is unsubstituted phenyl or phenyl substituted by (C₁₋₄)alkyl, (C₁₋₄)alkoxy or halogen, and
A⁻ is as defined above,
   in the preparation of a medicament for the treatment of a condition mediated by CCR3.

The compounds of the present invention act as CCR3 receptor antagonists, thereby inhibiting the infiltration and activation of inflammatory cells, particularly eosinophils, and inhibiting allergic response. The inhibitory properties of the compounds of the present invention can be demonstrated in the following assay:

In this assay the effect of the compounds of the present invention on the binding of human eotaxin to human CCR-3 is determined. Recombinant cells expressing human CCR3 are captured by wheatgerm agglutinin (WGA) polyvinyltoluidene (PVT) SPA beads (available from Amersham), through a specific interaction between the WGA and carbohydrate residues of glycoproteins on the surface of the cells. [¹²⁵I]-human eotaxin (available from Amersham) binds specifically to CCR3 receptors bringing the [¹²⁵I]-human eotaxin in close proximity to the SPA beads. Emitted α-particles from the [¹²⁵I]-human eotaxin excite, by its proximity, the fluorophore in the beads and produce light. Free [¹²⁵I]-human eotaxin in solution is not in close proximity to the scintillant and hence does not produce light. The scintillation count is therefore a measure of the extent to which the test compound inhibits binding of the eotaxin to the CCR3.

Preparation of Assay Buffer: 5.96 g HEPES and 7.0 g sodium chloride are dissolved in distilled H₂O and 1M aqueous CaCl₂ (1 ml) and 1M aqueous MgCl₂ (5 ml) are added. The pH is adjusted to 7.6 with NaOH and the solution made to a final volume of 1 L using distilled H₂O. 5 g of bovine serum albumin and 0.1 g NaN₃ are dissolved in the solution and the resulting buffer stored at 4° C. A Complete™ protease inhibitor cocktail tablet (available from Boehringer) is added per 50 ml of the buffer on the day of use.

Preparation of Homogenisation Buffer: Tris-base (2.42 g) is dissolved in distilled H₂O, the pH of the solution is adjusted to 7.6 with HCl and the solution obtained is diluted with distilled H₂O to a final volume of 1 l. The resulting buffer is stored at 4° C. A Complete™ protease inhibitor cocktail tablet is added per 50 ml of the buffer on the day of use.

Preparation of membranes: Confluent rat basophil leukaemia (RBL-2H3) cells stably expressing CCR3 are removed from tissue culture flasks using enzyme-free cell dissociation buffer and resuspended in phosphate-buffered saline. The cells are centrifuged (800 g, 5 minutes), the pellet obtained is resuspended in ice-cold homogenisation buffer using 1 ml homogenisation buffer per gram of cells and incubated on ice for 30 minutes. The cells are homogenised on ice with 10 strokes in a glass mortar and pestle. The homogenate is centrifuged (800 g, 5 minutes, 4° C.), the supernatant obtained is centrifuged (48,000 g, 30 minutes, 4° C.) and the pellet obtained is redissolved in Homogenisation Buffer containing 10% (v/v) glycerol. The protein content of the membrane preparation is estimated by the method of Bradford (Anal. Biochem. (1976) 72:248) and aliquots are snap frozen and stored at −80° C.

The assay is performed in a final volume of 250 μl per well of an Optiplate™ microplate (ex Canberra Packard). 50 μl of solutions of a test compound in Assay Buffer containing 5% DMSO (concentrations from 0.01 nM to 10 μM) are added to selected wells of the microplate. To determine total binding, 50 μl of the Assay Buffer containing 5% DMSO is added to other selected wells. To determine non-specific binding, 50 μl of 100 nM human eotaxin (ex R&D Systems) in Assay Buffer containing 5% DMSO is added to further selected wells. 50 μl of [¹²⁵I]-Human eotaxin (ex Amersham) in Assay Buffer containing 5% DMSO at a concentration of 250 μM (to give a final concentration of 50 μM per well), 50 μl of WGA-PVT SPA beads in Assay Buffer (to give a final concentration of 1.0 mg beads per well) and 100 μl of the membrane preparation at a concentration of 100 μg protein in Assay Buffer (to give a final concentration of 10 μg protein per well) are added to all wells. The plate is then incubated for 4 hours at room temperature. The plate is sealed using TopSeal-S™ sealing tape (ex Canberra Packard) according to the manufacturer's instructions. The resulting scintillations are counted using a Canberra Packard TopCount™ scintillation counter, each well being counted for 1 minute. The concentration of test compound at which 50% inhibition occurs (IC₅₀) is determined from concentration-inhibition curves in a conventional manner.

The compounds of the Examples herein below generally have IC₅₀ values below 1 μM in the above assay. For instance, the compound of example 241 has an IC₅₀ value of 6 nM, the compound of example 322 has an IC₅₀ value of 21 nM and the compound of example 341 has an IC₅₀ value of 23 nM.

Most of the compounds of the Examples exhibit selectivity for inhibition of CCR3 binding relative to inhibition of binding of the alpha-1 adrenergic receptor.

The inhibitory properties of the compounds of the present invention on binding of the alpha-1 adrenergic receptor can be determined in the following assay:

Cerebral cortices from male Sprague-Dawley rats (175-200 g) are dissected and homogenised in 10 volumes of ice cold 0.32 M sucrose (containing 1 mM MgCl₂ dihydrate and 1 mM K₂HPO₄) with a glass/Teflon homogeniser. The membranes are centrifuged at 1000×g for 15 minutes, the pellet discarded and the centrifugation repeated. The supernatants are pooled and centrifuged at 18,000×g for 15 minutes. The pellet is osmotically shocked in 10 volumes of H₂O and kept on ice for 30 minutes. The suspension is centrifuged at 39,000×g for 20 minutes, resuspended in Krebs-Henseleit buffer pH 7.4 (1.17 mM MgSO₄ anhydrous, 4.69 mM KCl, 0.7 mM K₂HPO₄ anhydrous, 0.11 M NaCl, 11 mM D-glucose and 25 mM NaHCO₃) containing 20 mM Tris, and kept for 2 days at −20° C. The membranes are thawed at 20-23° C., washed three times with Krebs-Henseleit buffer by centrifugation at 18,000×g for 15 minutes, left overnight at 4° C. and washed again 3 times. The final pellet is resuspended with a glass/Teflon homogeniser in 125 ml/100 membranes in the same buffer. A sample is taken to determine the protein concentration (using the Bradford Assay with gamma globulin as the standard) and the remainder aliquoted and stored at −80° C.

The resulting membranes are subjected to a radioligand binding assay. The assay is conducted in triplicate using 96 well plates containing [¹²⁵I]-HEAT (Amersham) (40 pM, K_{d}: 58.9±18.7 pM), unlabelled test compound and membrane (57.1 μg/ml) to yield a final volume of 250 μl (assay buffer containing 50 mM Tris-base and 0.9% (w/v) NaCl, pH 7.4). The plates are incubated at 37° C. for 60 minutes, after which rapid vacuum filtration over Whatman™ GF/C 96 well filter plates is carried out. Each plate is then washed three times with 10 ml of ice cold assay buffer using a Brandel Cell harvester (Gaithersburg, Md.). Following drying of the plates for 3 h. at 50° C., 40 μl of Microscint 20 is added to each well, the plates incubated at room temperature for a further 20 minutes and the retained radioactivity quantified in a Packard TopCount NXT™ scintillation counter.

Stock solutions of test compounds are dissolved initially in 100% DMSO and diluted with assay buffer to the required concentrations to yield 1% (v/v) DMSO. The concentration of test compound at which 50% inhibition occurs (IC₅₀) is determined from concentration-inhibition curves in a conventional manner.

Having regard to their inhibition of binding of CCR3, the compounds of the present invention are useful in the treatment of conditions mediated by CCR3, particularly inflammatory or allergic conditions. Treatment in accordance with the present invention may be symptomatic or prophylactic.

Accordingly, compounds of the present invention are useful in the treatment of inflammatory or obstructive airways diseases, resulting, for example, in reduction of tissue damage, bronchial hyper-reactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial or viral infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), acute/adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The present invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, compounds of the present invention are also useful in the treatment of eosinophil related disorders, e.g. eosinophilia, in particular eosinophil related disorders of the airways (e.g. involving morbid eosinophilic infiltration of pulmonary tissues) including hyper-eosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

The compounds of the present invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

The compounds of the present invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, e.g. atrophic, chronic, or seasonal rhinitis, inflammatory conditions of the gastrointestinal tract, for example inflammatory bowel disease such as ulcerative colitis and Crohn's disease, diseases of the bone and joints including rheumatoid arthritis, psoriatic arthritis, ankylosing spondylitis and systemic sclerosis, and other diseases such as cyctic fibrosis, pulmonary hypertension, atherosclerosis, multiple sclerosis, diabetes (type I), myasthenia gravis, hyper IgE syndrome and acute and chronic allograft rejection, e.g. following transplantation of heart, kidney, liver, lung or bone marrow.

The effectiveness of a compound of the present invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The compounds of the present invention are also useful as co-therapeutic compounds for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the present invention may be mixed with the other drug substance in a fixed pharmaceutical composition or it may be administered separately, before, simultaneously with or after the other drug substance.

Such anti-inflammatory drugs include steroids, in particular glucocorticosteroids such as budesonide, beclamethasone, fluticasone, ciclesonide or mometasone, or steroids described in WO 02/88167, WO 02/12266, WO 02/100879, WO 04/039827 or WO 02/00679, especially those of Examples 3, 11, 14, 17, 19, 26, 34, 37, 39, 51, 60, 67, 72, 73, 90, 99 and 101; LTB4 antagonists such as those described in U.S. Pat. No. 5,451,700, also LY293111, CGS025019C, CP-195543, SC-53228, BIIL 284, ONO 4057 and SB 209247; LTD4 antagonists such as montelukast and zafirlukast; Dopamine receptor agonists such as cabergoline, bromocriptine, ropinirole and 4-hydroxy-7-[2-[[2-[[3-(2-phenylethoxy)propyl]sulfonyl]ethyl]-amino]ethyl]-2(3H)-benzothiazolone and pharmaceutically acceptable salts thereof (the hydrochloride being Viozane®-AstraZeneca); PDE4 inhibitors such as cilomilast (Ariflo® GSK), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(TM) CC-10004 (Celgene), VM554/UM565 (Vernalis), T-440 (Tanabe), KW4490 (Kyowa Hakko Kogyo), WO 92/19594, WO 93/19749, WO 93/19750, WO 93/19751, WO 99/16766, WO 01/13953, WO 03/104204, WO 03/104205, WO 04/000814, WO 04/000839 and WO 04/005258, WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945 and WO 04/045607, WO 04/037805 as well as those described in WO 98/18796 and WO 03/39544; A2a agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618, WO 04/046083; and A2b antagonists such as those described in WO 02/42298.

Such bronchodilatory drugs include anticholinergic or antimuscarinic agents, in particular ipratropium bromide, oxitropium bromide, tiotropium bromide, CHF 4226 (Chiesi) and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, U.S. Pat. No. 5,171,744, WO 03/33495 and WO 04/018422; and beta (β)-2-adrenoceptor agonists such as albuterol (salbutamol), metaproterenol, terbutaline, salmeterol, fenoterol, procaterol, and especially, formoterol and pharmaceutically acceptable salts thereof, and compounds (in free or salt or solvate form) of formula (I) of WO 00/75114, which document is incorporated herein by reference, preferably compounds of the Examples thereof, especially a compound of formula

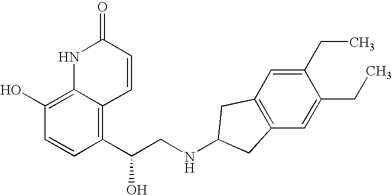

and pharmaceutically acceptable salts thereof, as well as compounds (in free or salt or solvate form) of formula (I) of WO 04/16601. Further suitable β-2-adrenoreceptor agonists include compounds such as those described in JP 05025045, US 2002/0055651, WO 93/18007, WO 99/64035, WO 01/42193, WO 01/83462, WO 02/066422, WO 02/070490, WO 02/076933, WO 03/24439, WO 03/72539, WO 03/42160, WO 03/91204, WO 03/42164, WO 03/99764, WO 04/11416, WO 04/16578, WO 04/22547, WO 04/32921, WO 04/33412, WO 04/37773, WO 04/37807, WO 04/39762, WO 04/39766, WO 04/45618 and WO 04/46083. Such co-therapeutic antihistamine drug substances include cetirizine hydrochloride, acetamino-phen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/99807 and WO 04/26841. Combinations of compounds of the present invention and one or more steroids, beta-2 agonists, PDE4 inhibitors or LTD4 antagonists may be used, for example, in the treatment of COPD or, particularly, asthma. Combinations of compounds of the present invention and anticholinergic or antimuscarinic agents, PDE4 inhibitors, dopamine receptor agonists or LTB4 antagonists may be used, for example, in the treatment of asthma or, particularly, COPD. Other useful combinations of compounds of the present invention with anti-inflammatory drugs are those with other antagonists of chemokine receptors, e.g. CCR1, CCR2, CCR4, CCR5, CCR6, CCR7, CCR8, CCR9 and CCR10, CXCR1, CXCR2, CXCR3, CXCR4, CXCR5, particularly CCR5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770), CCR5 antagonists described in U.S. Pat. No. 6,166,037 (particularly claims 18 and 19), WO 00/66558 (particularly claim 8), and WO 00/66559 (particularly claim 9), WO 04/018425 and WO 04/026873.

In accordance with the foregoing, the present invention also provides a method for the treatment of a condition mediated by CCR3, for example an inflammatory or allergic condition, particularly an inflammatory or obstructive airways disease, which comprises administering to a subject, particularly a human subject, in need thereof an effective amount of a compound of formula (I) in a free or pharmaceutically acceptable salt form as hereinbefore described.

In another aspect the present invention provides the use of a compound of formula (I), in free or pharmaceutically acceptable salt form, as hereinbefore described for the manufacture of a medicament for the treatment of a condition mediated by CCR3, e.g. an inflammatory or allergic condition, particularly an inflammatory or obstructive airways disease.

The compounds of the present invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, e.g. in the treatment of atopic dermatitis; or rectally, e.g. in the treatment of inflammatory bowel disease.

In a further aspect, the present invention also provides a pharmaceutical composition comprising as active ingredient a compound of formula I in free or pharmaceutically acceptable salt form, optionally together with a pharmaceutically acceptable diluent or carrier therefor. The composition may contain a co-therapeutic agent such as an anti-inflammatory bronchodilatory or antihistamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula (I) having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it preferably contains, for example, the compound of formula (I) either dissolved, or suspended, in a vehicle containing H₂O, a co-solvent such as EtOH or propylene glycol and a stabiliser, which may be a surfactant.

The present invention includes (A) a compound of the present invention in inhalable form, e.g. in an aerosol or other atomisable composition or in inhalable particulate, e.g. micronised form, (B) an inhalable medicament comprising a compound of the present invention in inhalable form; (C) a pharmaceutical product comprising such a compound of the present invention in inhalable form in association with an inhalation device; and (D) an inhalation device containing a compound of the present invention in inhalable form.

Dosages of compounds of the present invention employed in practising the present invention will of course vary depending, for example, on the particular condition to be treated, the effect desired and the mode of administration. In general, suitable daily dosages for administration by inhalation are of the order of 0.01 to 30 mg/kg while for oral administration suitable daily doses are of the order of 0.01 to 100 mg/kg.

| EXAMPLE | R₁ | R₂ |
|---|---|---|
| 1 | 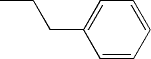 | 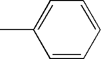 |
| 2 | 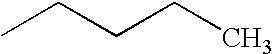 | 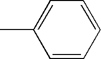 |
| 3 | 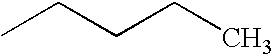 | 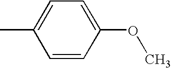 |
| 4 | 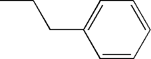 | 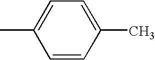 |
| 5 | 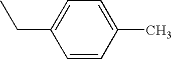 | 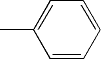 |
| 6 | 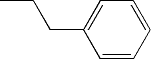 | 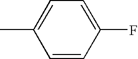 |
| 7 | 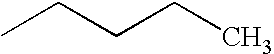 | 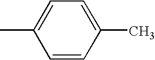 |
| 8 | 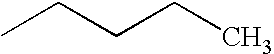 | 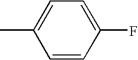 |
| 9 | 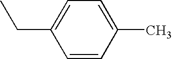 | 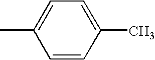 |
| 10 | 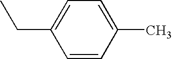 | 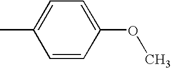 |
| 11 | 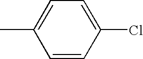 | 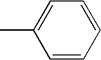 |
| 12 | 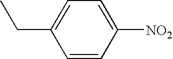 | 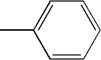 |
| 13 | 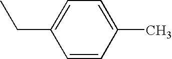 | 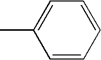 |
| 14 | 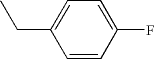 | 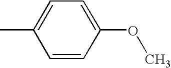 |
| 15 | 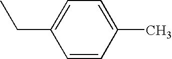 | 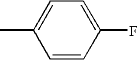 |
| 16 | 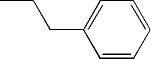 | 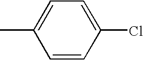 |
| 17 | 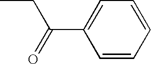 | 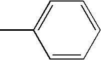 |
| 18 | 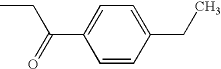 | 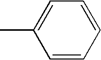 |
| 19 | 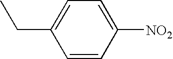 | 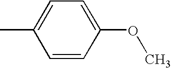 |
| 20 | 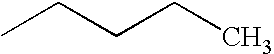 | 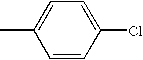 |
| 21 | 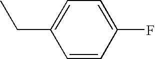 | 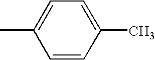 |
| 22 | 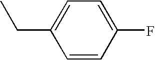 | 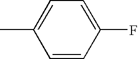 |
| 23 | 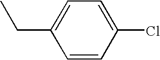 | 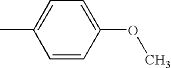 |
| 24 | 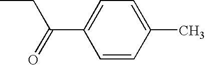 | 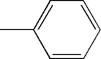 |
| 25 | 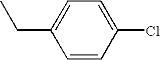 | 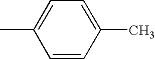 |
| 26 | 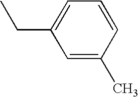 | 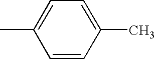 |
| 27 | 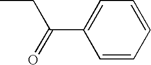 | 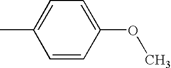 |
| 28 | 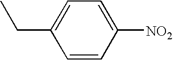 | 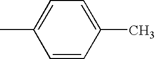 |
| 29 | 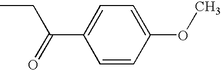 | 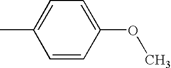 |
| 30 | 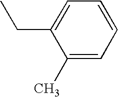 | 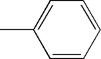 |
| 31 | 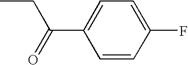 | 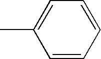 |
| 32 | 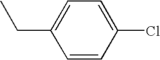 | 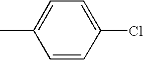 |
| 33 | 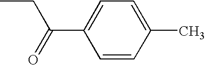 | 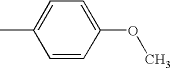 |
| 34 | 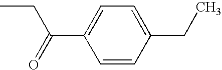 | 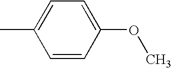 |
| 35 | 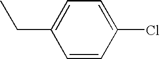 | 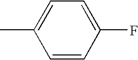 |
| 36 | 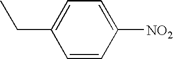 | 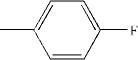 |
| 37 | 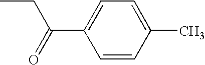 | 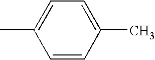 |
| 38 | 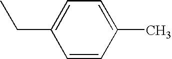 | 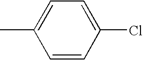 |
| 39 | 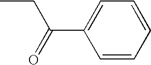 | 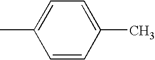 |
| 40 | 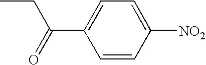 | 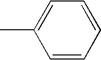 |
| 41 | 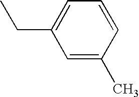 | 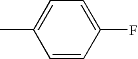 |
| 42 | 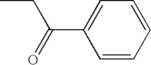 | 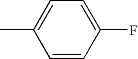 |
| 43 | 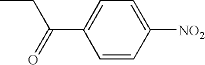 | 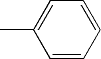 |
| 44 | 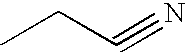 | 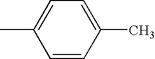 |
| 45 | 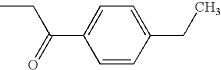 | 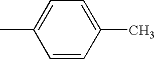 |
| 46 | 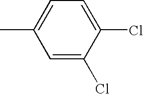 | 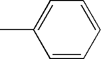 |
| 47 | 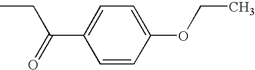 | 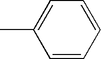 |
| 48 | 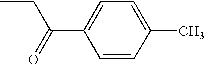 | 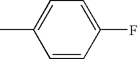 |
| 49 | 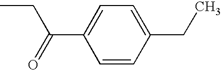 | 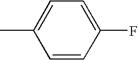 |
| 50 | 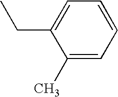 | 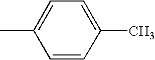 |
| 51 | 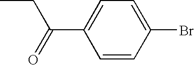 | 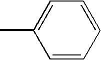 |
| 52 | 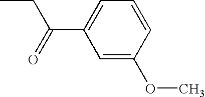 | 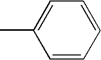 |
| 53 | 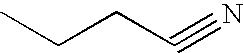 | 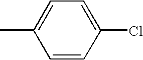 |
| 54 | 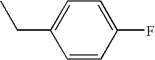 | 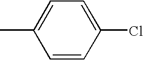 |
| 55 | 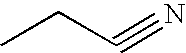 | 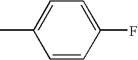 |
| 56 | 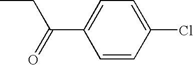 | 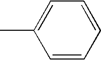 |
| 57 | 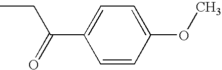 | 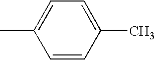 |
| 58 | 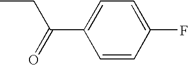 | 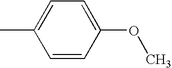 |
| 59 | 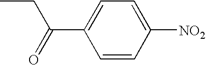 | 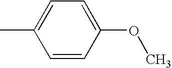 |
| 60 | 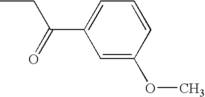 | 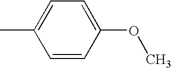 |
| 61 | 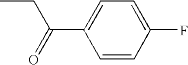 | 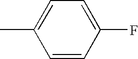 |
| 62 | 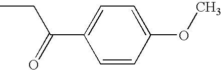 | 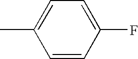 |
| 63 | 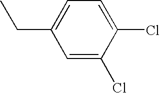 | 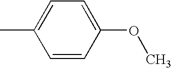 |
| 64 | 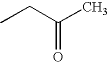 | 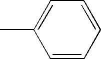 |
| 65 | 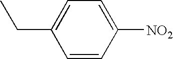 | 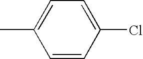 |
| 66 | 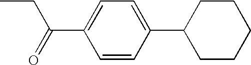 | 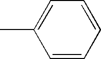 |
| 67 | 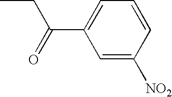 | 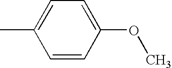 |
| 68 | 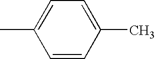 | 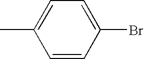 |
| 69 | 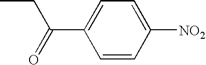 | 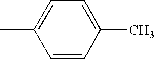 |
| 70 | 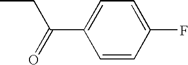 | 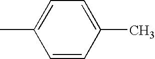 |
| 71 | 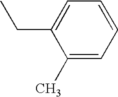 | 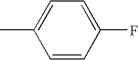 |
| 72 | 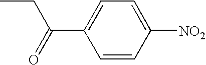 | 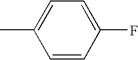 |
| 73 | 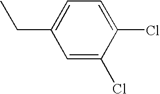 | 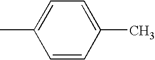 |
| 74 | 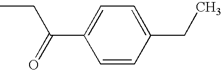 | 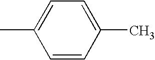 |
| 75 | 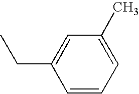 | 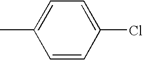 |
| 76 | 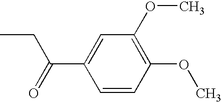 | 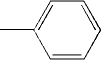 |
| 77 | 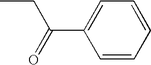 | 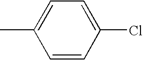 |
| 78 | 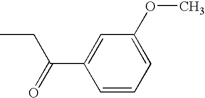 | 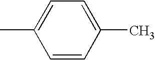 |
| 79 | 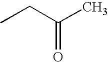 | 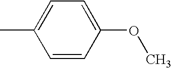 |
| 80 | 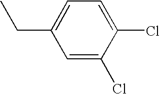 | 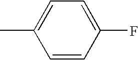 |
| 81 | 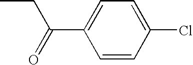 | 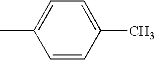 |
| 82 | 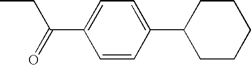 | 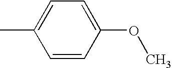 |
| 83 | 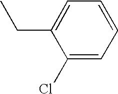 | 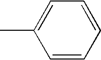 |
| 84 | 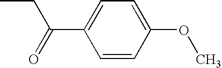 | 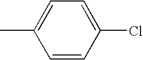 |
| 85 | 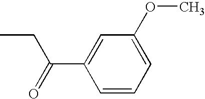 | 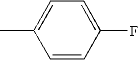 |
| 86 | 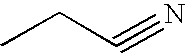 | 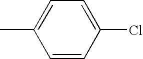 |
| 87 | 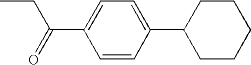 | 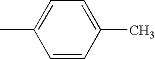 |
| 88 | 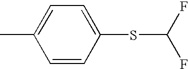 | 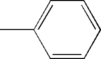 |
| 89 | 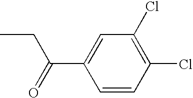 | 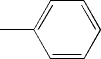 |
| 90 | 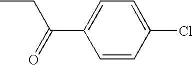 | 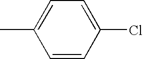 |
| 91 | 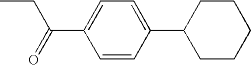 | 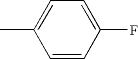 |
| 92 | 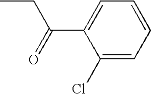 | 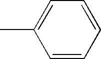 |
| 93 | 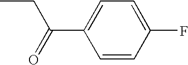 | 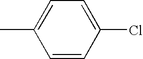 |
| 94 | 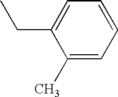 | 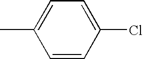 |
| 95 | 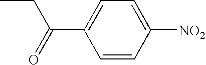 | 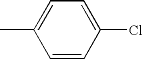 |
| 96 | 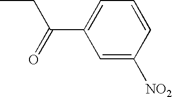 | 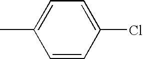 |
| 97 | 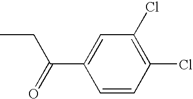 | 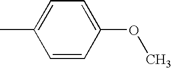 |
| 98 | 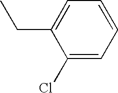 | 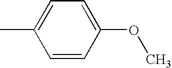 |
| 99 | 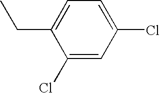 | 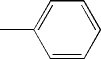 |
| 100 | 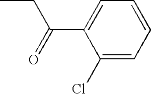 | 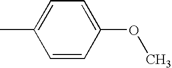 |
| 101 | 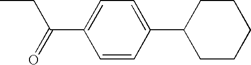 | 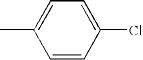 |
| 102 | 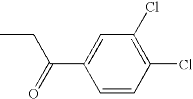 | 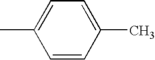 |
| 103 | 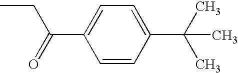 | 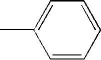 |
| 104 | 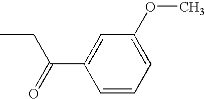 | 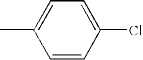 |
| 105 | 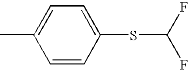 | 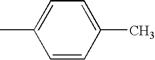 |
| 106 | 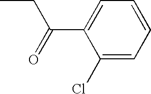 | 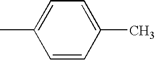 |
| 107 | 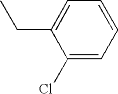 | 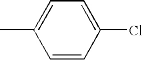 |
| 108 | 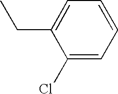 | 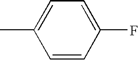 |
| 109 | 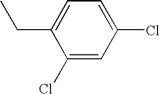 | 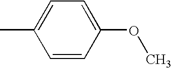 |
| 110 | 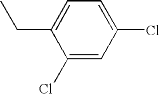 | 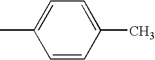 |
| 111 | 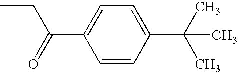 | 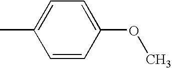 |
| 112 | 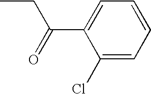 | 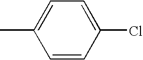 |
| 113 | 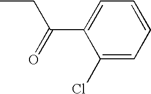 | 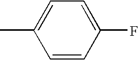 |
| 114 | 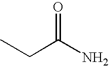 | 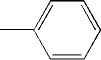 |
| 115 | 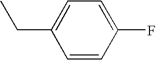 | 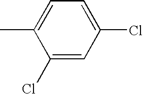 |
| 116 | 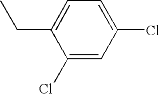 | 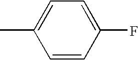 |
| 117 | 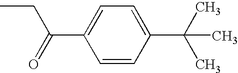 | 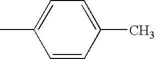 |
| 118 | 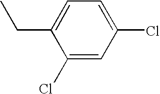 | 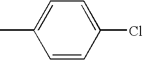 |
| 119 | 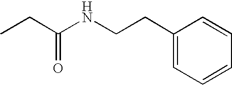 | 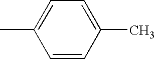 |
| 120 | 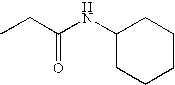 | 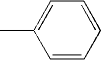 |
| 121 | 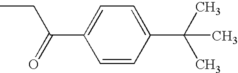 | 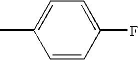 |
| 122 | 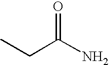 | 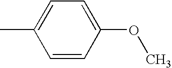 |
| 123 | 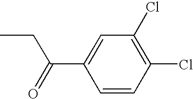 | 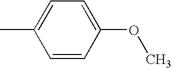 |
| 124 | 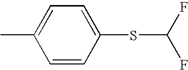 | 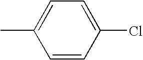 |
| 125 | 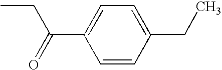 | 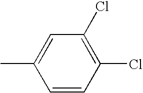 |
| 126 | 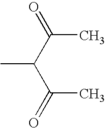 | 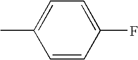 |
| 127 | 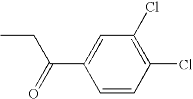 | 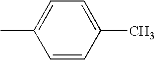 |
| 128 | 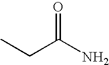 | 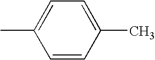 |
| 129 | 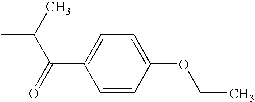 | 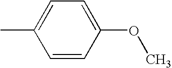 |
| 130 | 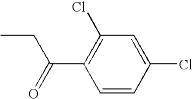 | 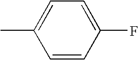 |
| 131 | 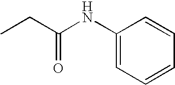 | 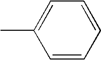 |
| 132 | 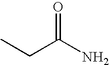 | 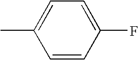 |
| 133 | 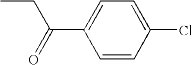 | 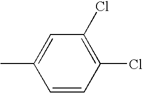 |
| 134 | 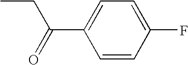 | 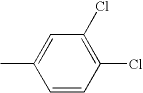 |
| 135 | 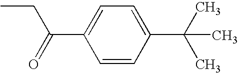 | 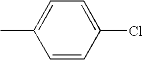 |
| 136 | 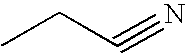 | 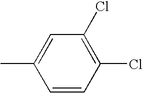 |
| 137 | 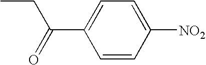 | 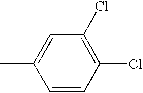 |
| 138 | 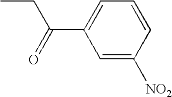 | 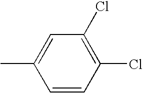 |
| 139 | 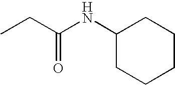 | 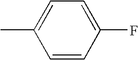 |
| 140 | 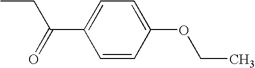 | 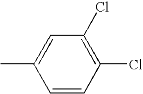 |
| 141 | 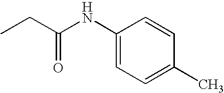 | 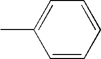 |
| 142 | 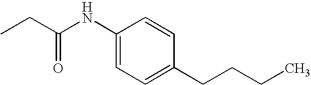 | 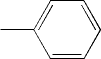 |
| 143 | 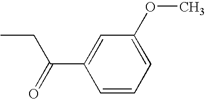 | 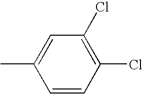 |
| 144 | 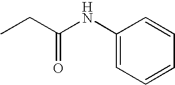 | 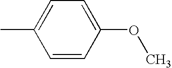 |
| 145 | 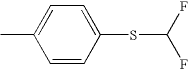 | 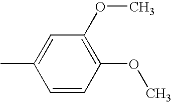 |
| 146 | 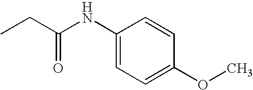 | 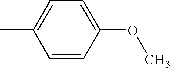 |
| 147 | 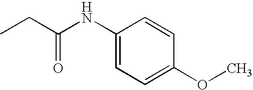 | 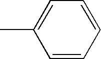 |
| 148 | 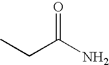 | 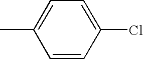 |
| 149 | 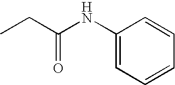 | 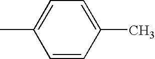 |
| 150 | 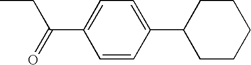 | 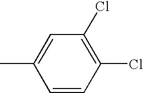 |
| 151 | 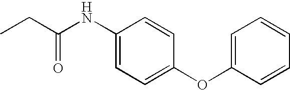 | 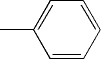 |
| 152 | 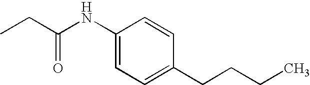 | 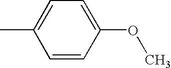 |
| 153 | 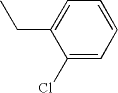 | 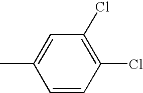 |
| 154 | 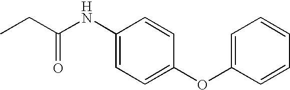 | 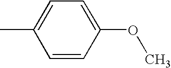 |
| 155 | 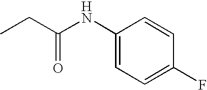 | 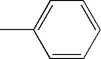 |
| 156 | 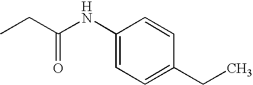 | 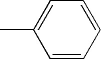 |
| 157 | 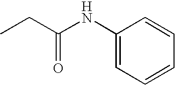 | 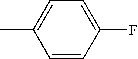 |
| 158 | 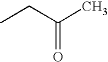 | 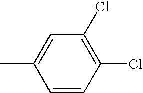 |
| 159 | 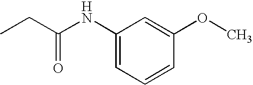 | 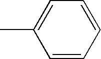 |
| 160 | 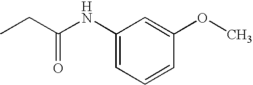 | 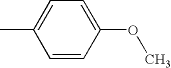 |
| 161 | 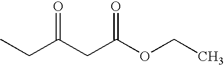 | 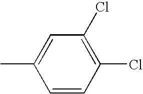 |
| 162 | 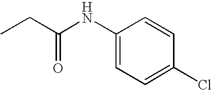 | 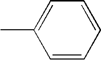 |
| 163 | 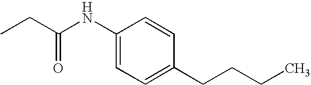 | 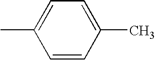 |
| 164 | 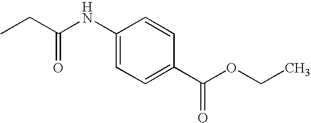 | 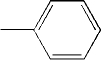 |
| 165 | 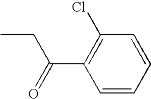 | 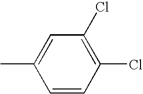 |
| 166 | 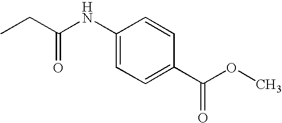 | 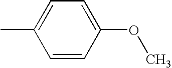 |
| 167 | 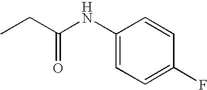 | 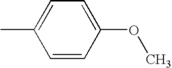 |
| 168 | 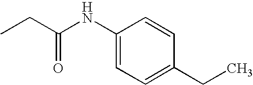 | 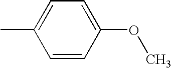 |
| 169 | 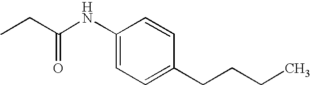 | 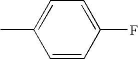 |
| 170 | 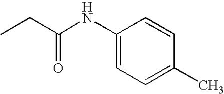 | 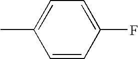 |
| 171 | 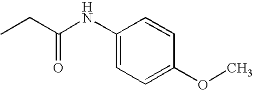 | 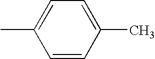 |
| 172 | 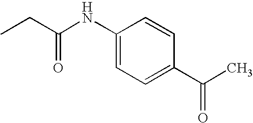 | 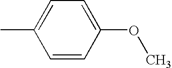 |
| 173 | 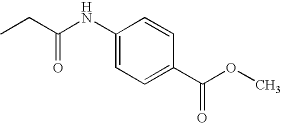 | 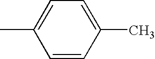 |
| 174 | 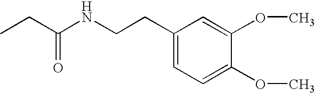 | 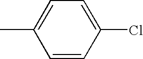 |
| 175 | 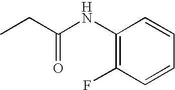 | 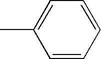 |
| 176 | 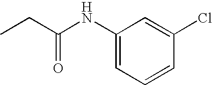 | 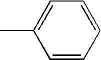 |
| 177 | 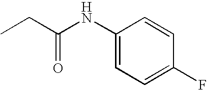 | 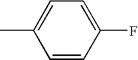 |
| 178 | 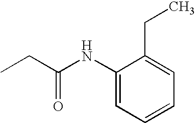 | 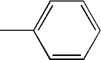 |
| 179 | 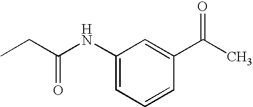 | 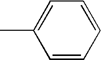 |
| 180 | 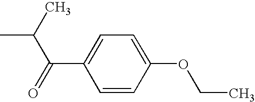 | 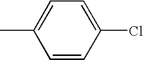 |
| 181 | 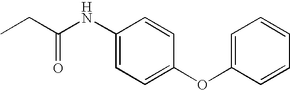 | 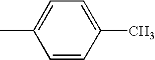 |
| 182 | 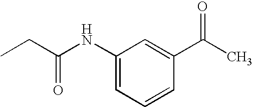 | 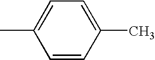 |
| 183 | 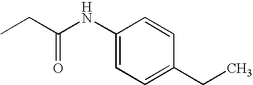 | 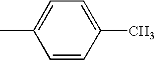 |
| 184 | 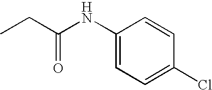 | 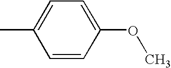 |
| 185 | 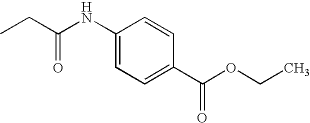 | 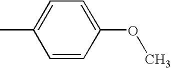 |
| 186 | 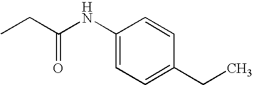 | 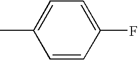 |
| 187 | 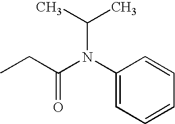 | 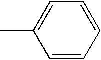 |
| 188 | 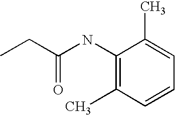 | 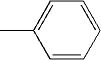 |
| 189 | 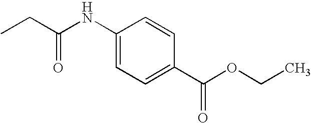 | 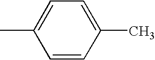 |
| 190 | 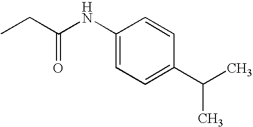 | 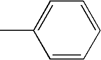 |
| 191 | 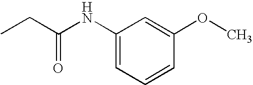 | 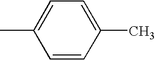 |
| 192 | 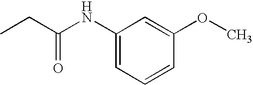 | 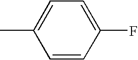 |
| 193 | 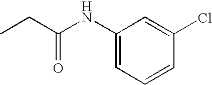 | 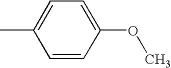 |
| 194 | 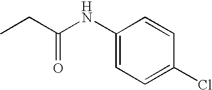 | 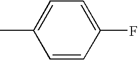 |
| 195 | 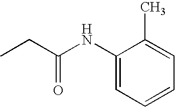 | 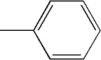 |
| 196 | 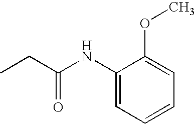 | 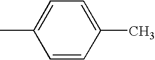 |
| 197 | 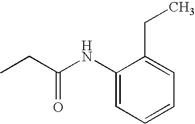 | 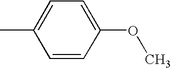 |
| 198 | 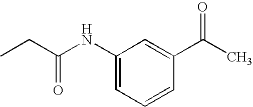 | 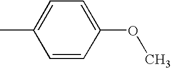 |
| 199 | 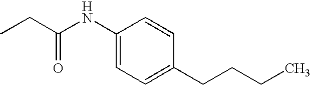 | 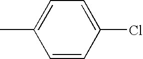 |
| 200 | 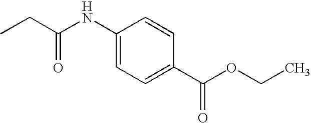 | 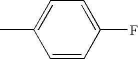 |
| 201 | 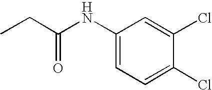 | 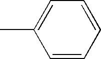 |
| 202 | 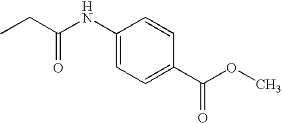 | 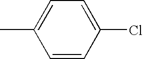 |
| 203 | 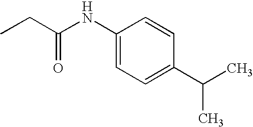 | 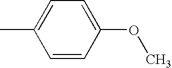 |
| 204 | 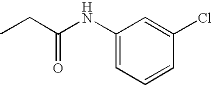 | 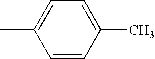 |
| 205 | 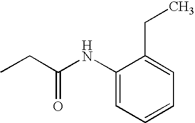 | 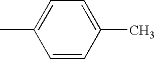 |
| 206 | 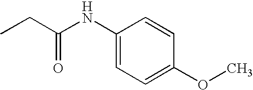 | 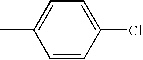 |
| 207 | 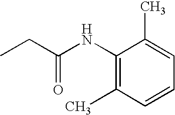 | 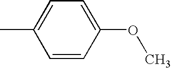 |
| 208 | 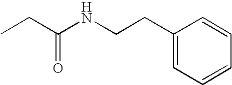 | 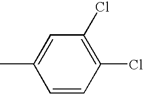 |
| 209 | 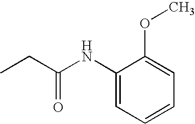 | 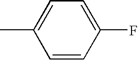 |
| 210 | 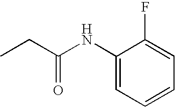 | 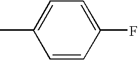 |
| 211 | 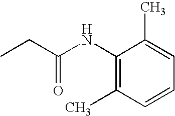 | 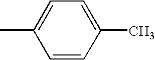 |
| 212 | 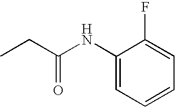 | 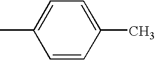 |
| 213 | 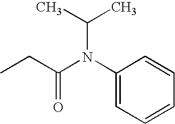 | 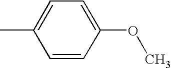 |
| 214 | 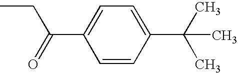 | 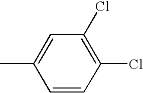 |
| 215 | 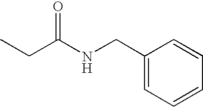 | 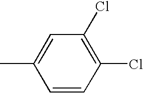 |
| 216 | 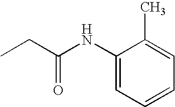 | 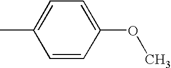 |
| 217 | 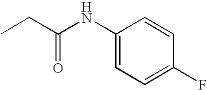 | 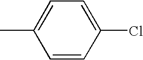 |
| 218 | 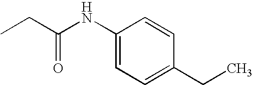 | 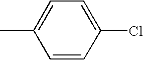 |
| 219 | 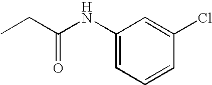 | 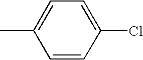 |
| 220 | 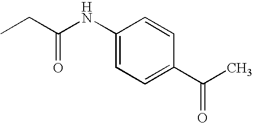 | 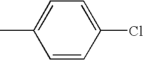 |
| 221 | 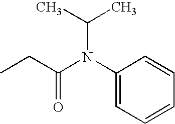 | 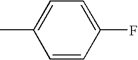 |
| 222 | 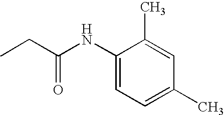 | 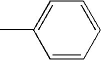 |
| 223 | 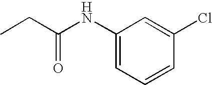 | 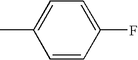 |
| 224 | 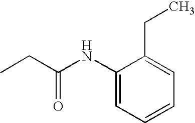 | 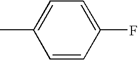 |
| 225 | 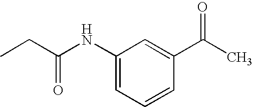 | 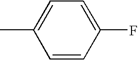 |
| 226 | 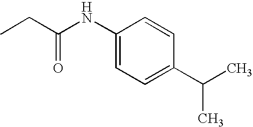 | 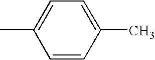 |
| 227 | 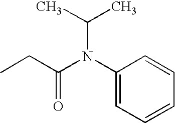 | 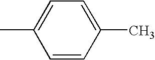 |
| 228 | 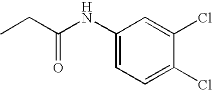 | 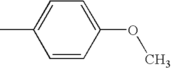 |
| 229 | 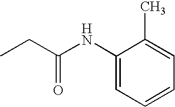 | 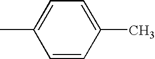 |
| 230 | 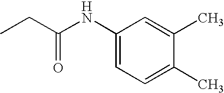 | 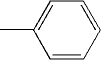 |
| 231 | 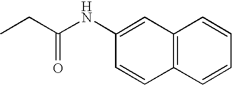 | 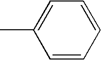 |
| 232 | 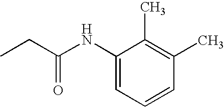 | 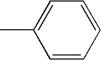 |
| 233 | 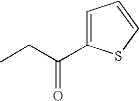 | 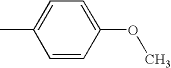 |
| 234 | 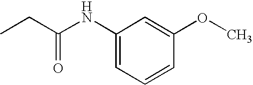 | 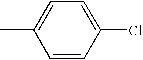 |
| 235 | 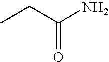 | 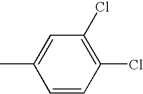 |
| 236 | 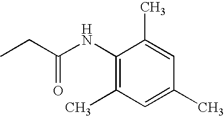 | 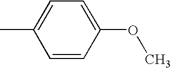 |
| 237 | 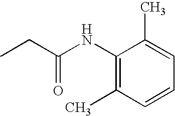 | 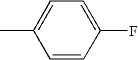 |
| 238 | 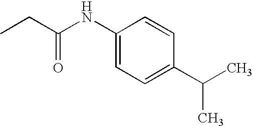 | 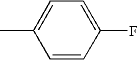 |
| 239 | 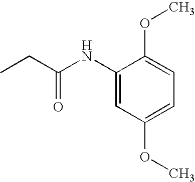 | 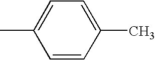 |
| 240 | 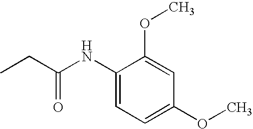 | 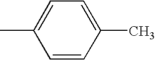 |
| 241 | 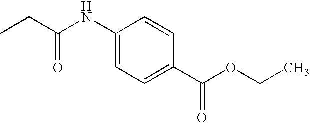 | 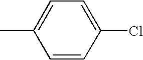 |
| 242 | 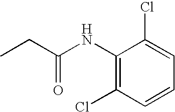 | 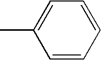 |
| 243 | 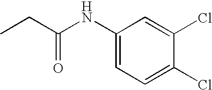 | 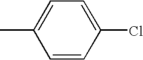 |
| 244 | 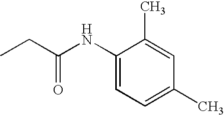 | 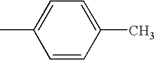 |
| 245 | 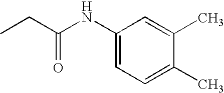 | 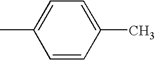 |
| 246 | 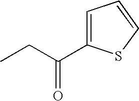 | 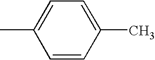 |
| 247 | 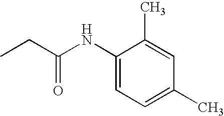 | 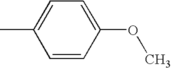 |
| 248 | 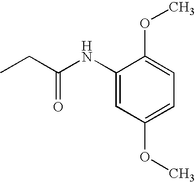 | 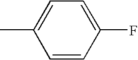 |
| 249 | 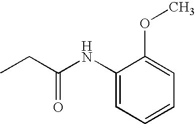 | 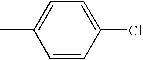 |
| 250 | 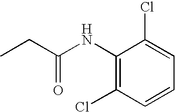 | 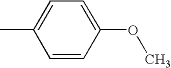 |
| 251 | 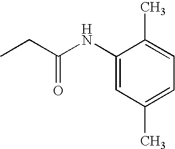 | 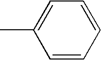 |
| 252 | 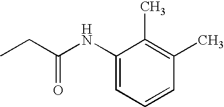 | 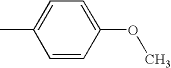 |
| 253 | 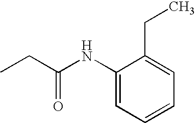 | 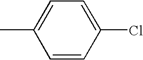 |
| 254 | 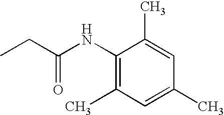 | 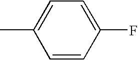 |
| 255 | 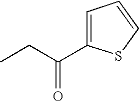 | 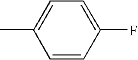 |
| 256 | 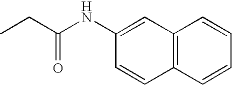 | 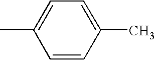 |
| 257 | 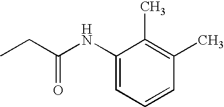 | 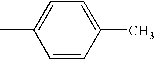 |
| 258 | 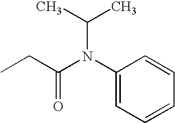 | 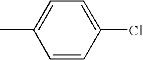 |
| 259 | 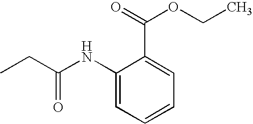 | 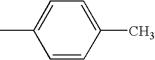 |
| 260 | 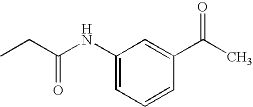 | 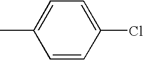 |
| 261 | 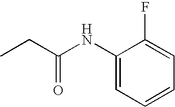 | 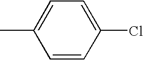 |
| 262 | 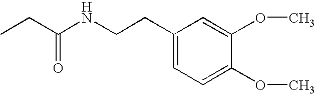 | 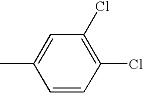 |
| 263 | 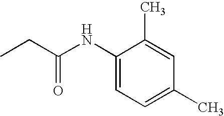 | 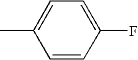 |
| 264 | 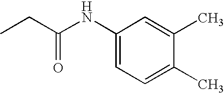 | 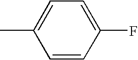 |
| 265 | 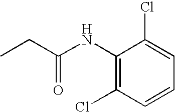 | 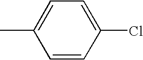 |
| 266 | 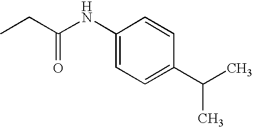 | 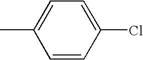 |
| 267 | 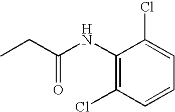 | 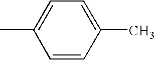 |
| 268 | 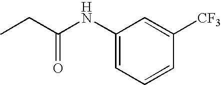 | 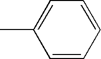 |
| 269 | 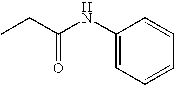 | 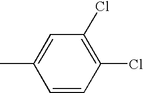 |
| 270 | 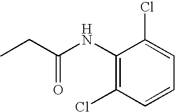 | 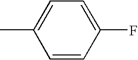 |
| 271 | 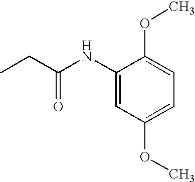 | 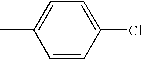 |
| 272 | 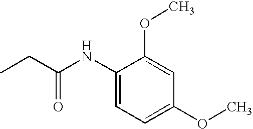 | 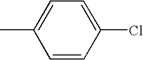 |
| 273 | 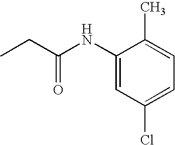 | 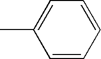 |
| 274 | 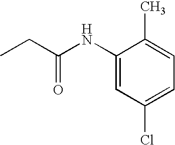 | 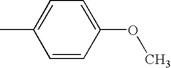 |
| 275 | 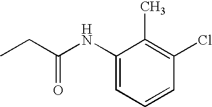 | 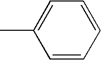 |
| 276 | 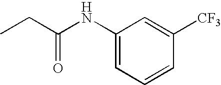 | 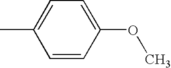 |
| 277 | 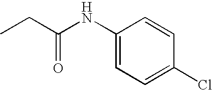 | 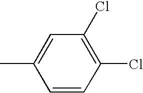 |
| 278 | 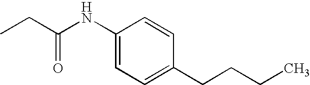 | 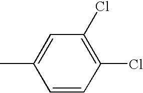 |
| 279 | 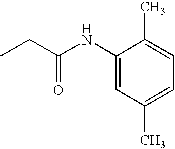 | 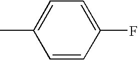 |
| 280 | 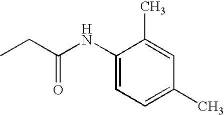 | 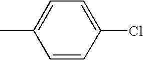 |
| 281 | 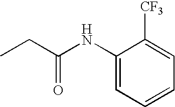 | 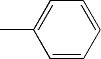 |
| 282 | 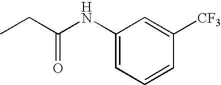 | 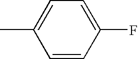 |
| 283 | 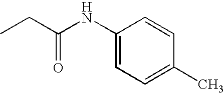 | 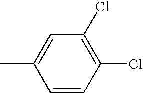 |
| 284 | 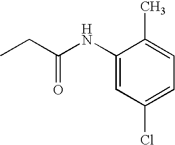 | 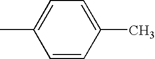 |
| 285 | 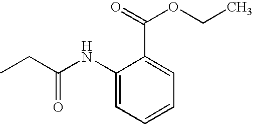 | 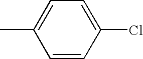 |
| 286 | 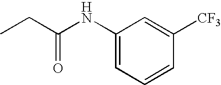 | 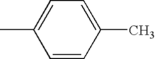 |
| 287 | 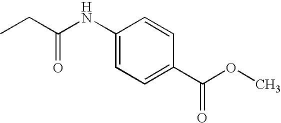 | 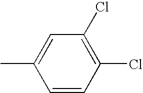 |
| 288 | 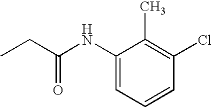 | 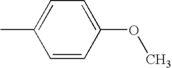 |
| 289 | 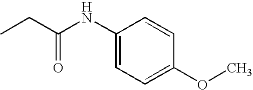 | 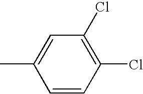 |
| 290 | 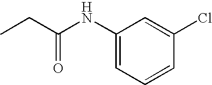 | 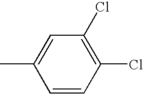 |
| 291 | 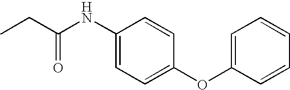 | 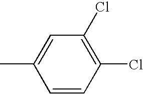 |
| 292 | 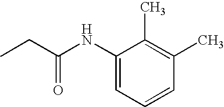 | 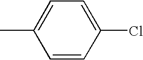 |
| 293 | 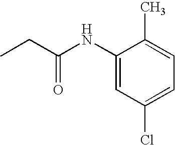 | 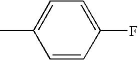 |
| 294 | 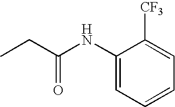 | 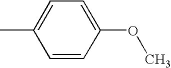 |
| 295 | 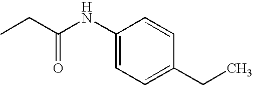 | 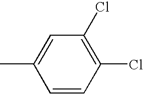 |
| 296 | 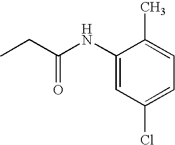 | 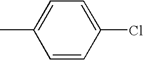 |
| 297 | 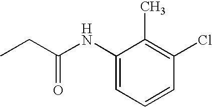 | 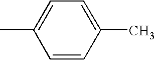 |
| 298 | 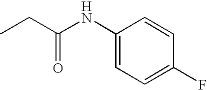 | 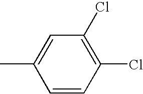 |
| 299 | 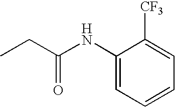 | 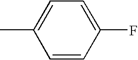 |
| 300 | 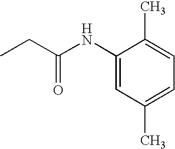 | 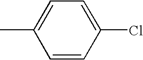 |
| 301 | 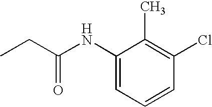 | 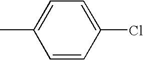 |
| 302 | 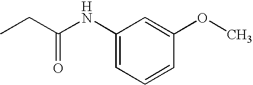 | 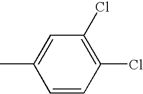 |
| 303 | 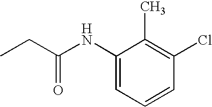 | 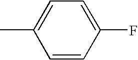 |
| 304 | 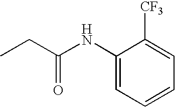 | 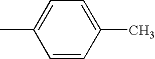 |
| 305 | 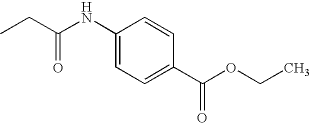 | 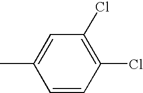 |
| 306 | 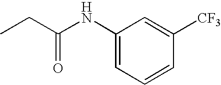 | 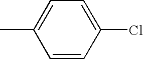 |
| 307 | 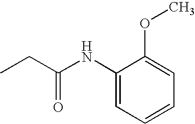 | 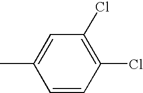 |
| 308 | 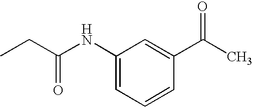 | 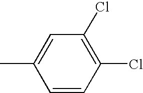 |
| 309 | 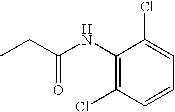 | 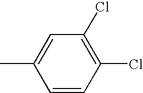 |
| 310 | 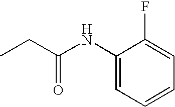 | 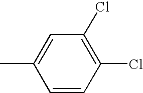 |
| 311 | 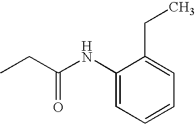 | 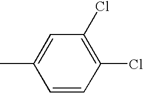 |
| 312 | 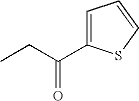 | 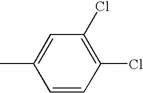 |
| 313 | 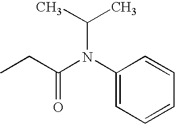 | 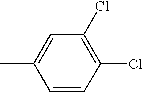 |
| 314 | 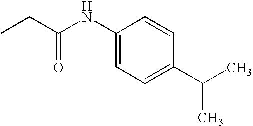 | 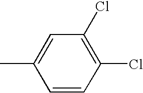 |
| 315 | 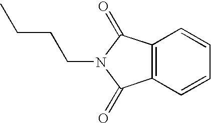 | 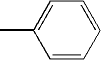 |
| 316 | 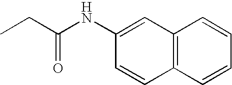 | 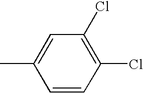 |
| 317 | 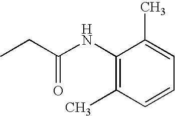 | 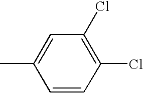 |
| 318 | 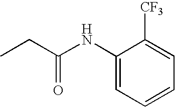 | 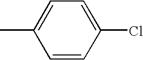 |
| 319 | 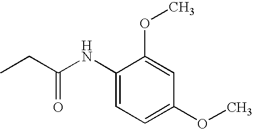 | 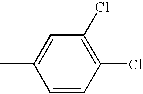 |
| 320 | 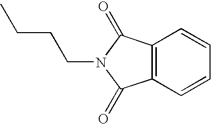 | 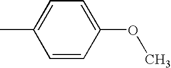 |
| 321 | 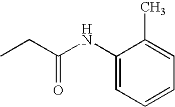 | 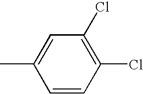 |
| 322 | 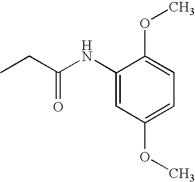 | 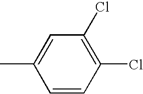 |
| 323 | 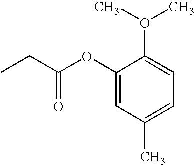 | 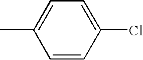 |
| 324 | 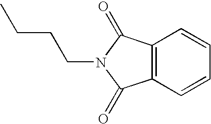 | 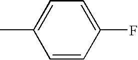 |
| 325 | 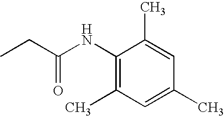 | 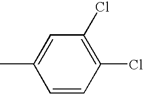 |
| 326 | 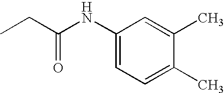 | 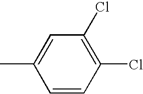 |
| 327 | 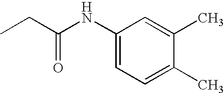 | 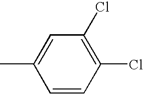 |
| 328 | 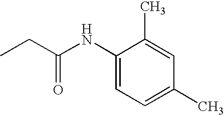 | 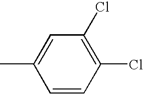 |
| 329 | 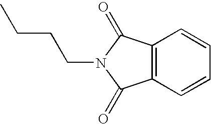 | 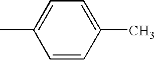 |
| 330 | 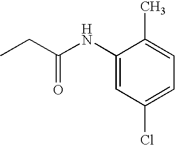 | 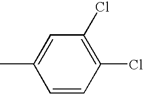 |
| 331 | 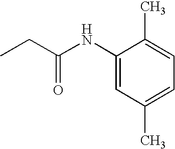 | 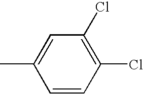 |
| 332 | 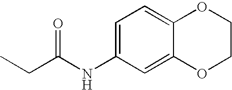 | 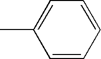 |
| 333 | 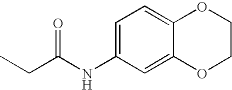 | 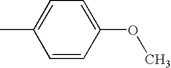 |
| 334 | 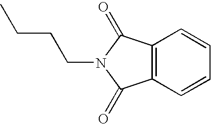 | 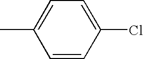 |
| 335 | 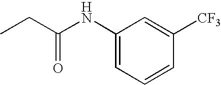 | 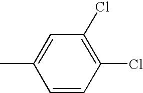 |
| 336 | 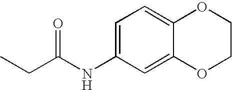 | 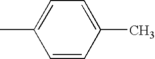 |
| 337 | 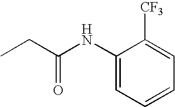 | 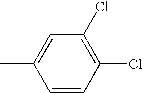 |
| 338 | 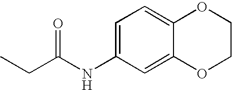 | 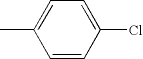 |
| 339 | 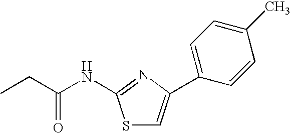 | 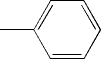 |
| 340 | 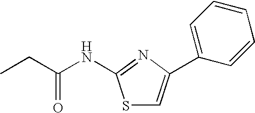 | 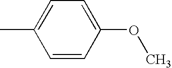 |
| 341 | 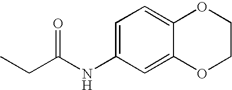 | 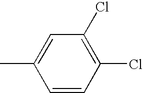 |
| 342 | 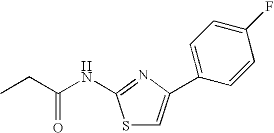 | 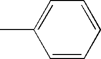 |
| 343 | 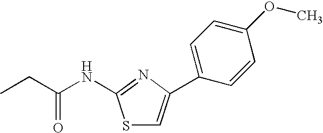 | 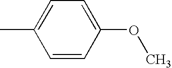 |
| 344 | 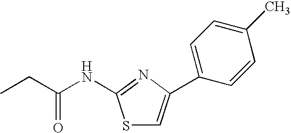 | 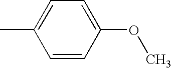 |
| 345 | 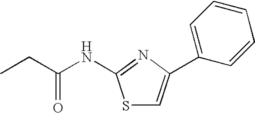 | 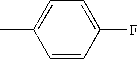 |
| 346 | 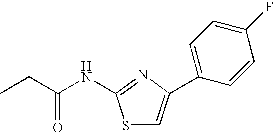 | 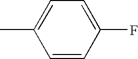 |
| 347 | 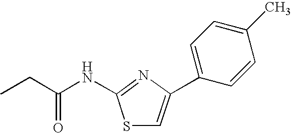 | 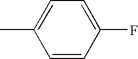 |
| 348 | 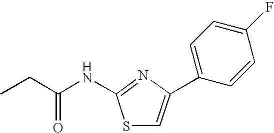 | 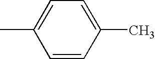 |
| 349 | 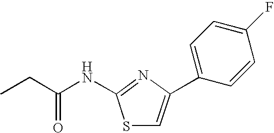 | 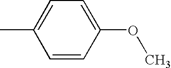 |
| 350 | 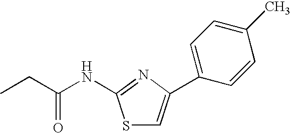 | 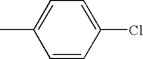 |
| 351 | 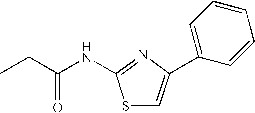 | 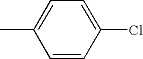 |
| 352 | 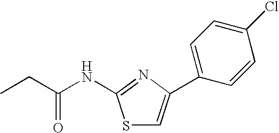 | 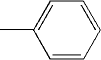 |
| 353 | 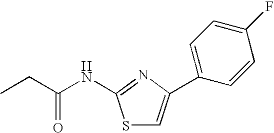 | 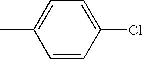 |
| 354 | 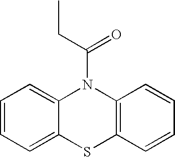 | 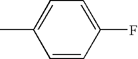 |
| 355 | 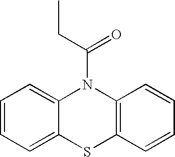 | 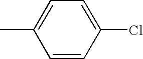 |
| 356 | 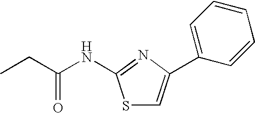 | 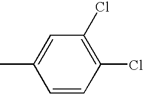 |
| 357 | 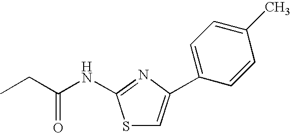 | 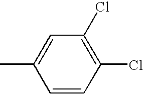 |
| 358 | 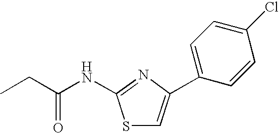 | 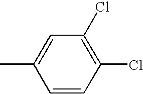 |
| 359 | 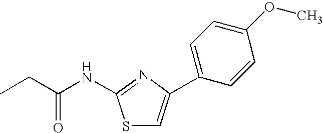 | 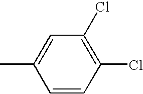 |
| 360 | 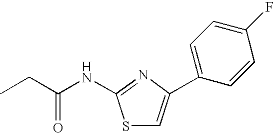 | 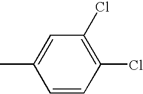 |
| 361 | 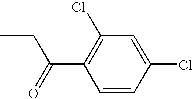 | 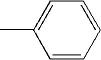 |
| 362 | 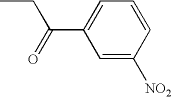 | 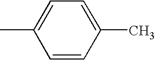 |
| 363 | 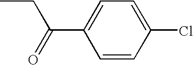 | 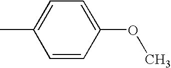 |
| 364 | 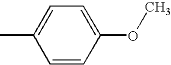 | 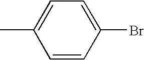 |
| 365 | 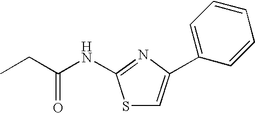 | 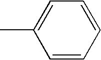 |
| 366 | 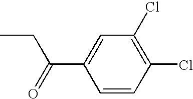 | 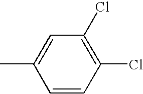 |
| 367 | 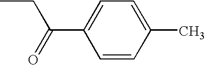 | 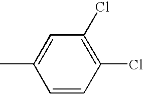 |
| 368 | 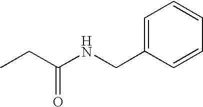 | 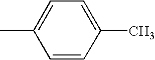 |
| 369 | 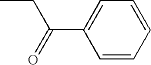 | 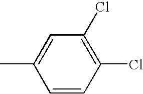 |
| 370AWL678 | 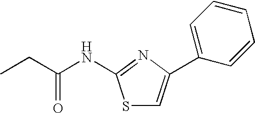 | 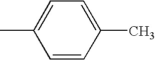 |
| 371 | 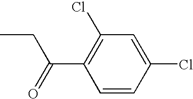 | 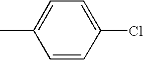 |
| 372 | 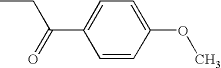 | 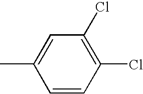 |
| 373 | 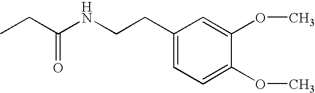 | 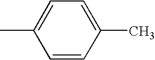 |
| 374 | 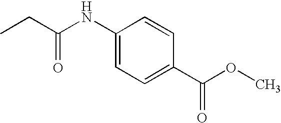 | 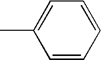 |
| 375 | 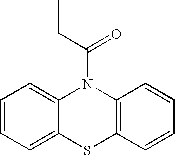 | 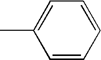 |
| 376 | 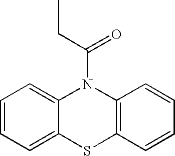 | 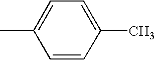 |
| 377 | 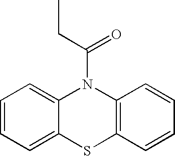 | 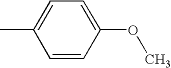 |
| 378 | 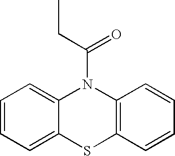 | 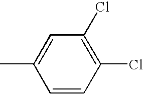 |

¹H-NMR spectra (DMSO-d₆, 400 MHz):

### EXAMPLE 239

9.88 (s, 1H), 7.98 (s, 1H), 7.68 (d, J=3 Hz, 1H), 7.55 (d, J=8 Hz, 2H), 7.35 (d, J=8 Hz, 2H), 7.00 (d, J=9 Hz, 1H), 6.67 (dd, J=8, 3 Hz, 1H), 5.22 (s, 2H), 4.50 (m, 2H), 3.81 (s, 3H), 3.56 (s, 3H), 3.20 (m, 2H), 2.73 (m, 2H), 2.36 (s, 3H)

### EXAMPLE 241

10.54 (s, 1H), 8.16 (s, 1H), 7.98 (d, J=2.1 Hz, 1H), 7.83 (d, J=8.4 Hz, 1H), 7.67 (dd, J=8.4, 2.1 Hz, 1H), 7.51 (d, J=10 Hz, 2H), 6.91 (d, J=10 Hz, 2H), 5.13 (s, 2H), 4.55 (m, 2H), 3.71 (s, 3H), 3.23 (m, 2H), 2.73 (m, 2H)

### EXAMPLE 322

9.90 (s, 1H), 8.16 (s, 1H), 7.98 (d, J=2.1 Hz, 1H), 7.83 (d, J=8.4 Hz, 1H), 7.66-7.71 (m, 2H), 7.00 (d, J=9.0 Hz, 1H), 6.67 (dd, J=9.0, 3.1 Hz, 1H), 5.24 (s, 2H), 4.55 (m, 2H), 3.82 (s, 3H), 3.65 (s, 3H), 3.22 (m, 2H), 2.73 (m, 2H)

### EXAMPLE 341

10.56 (s, 1H), 8.15 (s, 1H), 7.97 (d, J=2.1 Hz, 1H), 7.82 (d, J=8.4 Hz, 1H), 7.67 (dd, J=8.4, 2.1 Hz, 1H), 7.22 (d, J=2.5 Hz, 1H), 6.98 (dd, J=8.7, 2.5 Hz, 1H), 6.81 (d, J=8.7 Hz, 1H), 5.12 (s, 2H), 4.55 (m, 2H), 4.20 (m, 4H), 3.24 (m, 2H), 2.74 (m, 2H)

### EXAMPLE 373

8.52 (t, J=5.5 Hz, 1H), 7.88 (2, 1H), 7.54 (d, J=8.1 Hz, 2H), 7.35 (d, J=8.1 Hz, 2H), 6.85 (d, J=8.1 Hz, 1H), 6.81 (d, J=1.9 Hz, 1H), 6.72 (dd, J=8.1, 1.9 Hz, 1H), 4.87 (s, 2H), 4.48 (m, 2H), 3.73 (s, 3H), 3.69 (m, 3H), 3.34 (m, 2H), 3.09 (m, 2H), 2.65-2.73 (m, 4H), 2.35 (s, 3H)

1.-6. (canceled)

7. A method of treatment of a disease mediated by CCR3, which treatment comprises administering to a subject in need of such treatment a compound of formula (I) 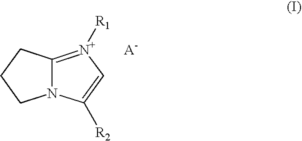
wherein
R₁ is—unsubstituted (C₁₋₆)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms:
(C₆₋₁₈)aryl, (C₆₋₁₈)aryl(C₁₋₆)alkyl, (C₆₋₁₈)aryl-carbonyl(C₁₋₆)alkyl, heterocyclyl, heterocyclyl(C₁₋₆)alkyl, heterocyclyl-carbonyl(C₁₋₄)alkyl, wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₁₋₆)alkyl, (C₁₋₄)alkoxy, (C₃₋₈)cycloalkyl, nitro, halo(C₁₋₄)alkyl or sulfanyl(C₁₋₄)alkyl;
aminocarbonyl(C₁₋₆)alkyl in unsubstituted form or one or morefold substituted by
(C₁₋₆)cycloalkyl,
halo(C₁₋₄)alkyl,
halogen,
unsubstituted (C₆₋₁₈)aryl,
(C₆₋₁₈)aryl substituted by (C₁₋₆)alkyl, (C₁₋₄)alkoxy, (C₃₋₈)cycloalkyl, nitro, halo(C₁₋₄)alkyl, halogen,
(C₆₋₈)aryl annelated with (C₆₋₁₈)aryl or heterocyclyl, wherein heterocyclyl has 5 or 6 ring members and 1 to 4 heteroatoms selected from N, O, S,
unsubstituted heterocyclyl, wherein heterocyclyl has 5 or 6 ring members and 1 to 4 heteroatoms selected from N, O, S.
heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₁₋₄)alkyl, halogen,
heterocyclyl annelated with (C₆₋₁₈)aryl or heterocyclyl, wherein heterocyclyl has 5 or 6 ring members and 1 to 4 heteroatoms selected from N, O, S.
R₂ is—unsubstituted (C₆₋₁₈ aryl or (C₆₋₁₈)aryl substituted by (C₁₋₄)alkyl, (C₁₋₄)alkoxy, (C₁₋₄)haloalkyl or halogen; and
A⁻ is a pharmaceutically acceptable anion.

8. A method of treatment of claim 7 wherein the disease is an inflammatory or allergic disease.

9. A method of claim 7, wherein the compound of formula (I) of is administered in combination with another pharmaceutically active agent, either simultaneously or sequentially.

10. A pharmaceutical composition comprising at least one pharmaceutical excipient and a compound of formula (I) 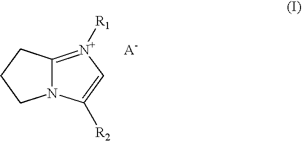
wherein
R₁ is—unsubstituted (C₁₋₆)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms;
(C₆₋₁₈)aryl, (C₆₋₁₈)aryl(C₁₋₆)alkyl, (C₆₋₁₈)aryl-carbonyl(C₁₋₄)alkyl, heterocyclyl, heterocyclyl(C₁₋₆)alkyl, heterocyclyl-carbonyl(C₁₋₆)alkyl wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₁₋₆)alkyl, (C₁₋₄)alkoxy, (C₃₋₈)cycloalkyl, nitro, halo(C₁₋₄)alkyl or sulfanyl(C₁₋₄)alkyl;
aminocarbonyl(C₁₋₆)alkyl in unsubstituted form or one or morefold substituted by
(C₁₋₆)alkyl;
(C₃₋₈)cycloalkyl;
halo(C₁₋₄)alkyl,
halogen,
unsubstituted (C₁₋₄)aryl,
(C₆₋₁₈)aryl substituted by (C₁₋₆)alkyl, (C₁₋₄)alkoxy. (C₃₋₈)cycloalkyl, nitro, halo(C₁₋₄)alkyl, halogen,
(C₆₋₁₈)aryl annelated with (C₆₋₁₈)aryl or heterocycyl, wherein heterocyclyl has 5 or 6 ring members and 1 to 4 heteroatoms selected from N, O, S,
unsubstituted heterocyclyl, wherein heterocyclyl has 5 or 6 ring members and 1 to 4 heteroatoms selected from N, O, S,
heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₁₋₄)alkyl, halogen,
heterocyclyl annelated with (C₆₋₁₈)aryl or heterocyclyl, wherein heterocyclyl has 5 or 6 ring members and 1 to 4 heteroatoms selected from N, O, S,
R₂ is—unsubstituted (C₆₋₁₈)aryl or (C₆₋₁₈)aryl substituted by (C₁₋₄)alkyl, (C₁₋₄)alkoxy, (C₁₋₄)haloalkyl or halogen; and
A⁻ is a pharmaceutically acceptable anion.

11. A pharmaceutical composition of claim 10 further comprising another pharmaceutically active agent.

12. The method of treatment of claim 7, wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl,
unsubstituted phenyl or phenyl one or morefold substituted by (C₁₋₄)alkyl, (C₁₋₂)alkoxy, halogen, nitro;
unsubstituted phenyl(C₁₋₄)alkyl or phenyl(C₁₋₄)alkyl one or morefold substituted by (C₁₋₄)alkyl, (C₁₋₂)alkoxy, nitro;
unsubstituted phenyl-carbonyl(C₁₋₂)alkyl or phenyl-carbonyl(C₁₋₂)alkyl one or morefold substituted by (C₁₋₄)alkyl, (C₁₋₂)alkoxy, halogen, nitro, (C₃₋₈)cycloalkyl, (C₁₋₄)alkyl-sulfanyl,
heterocyclyl-(C₁₋₄)alkyl, wherein heterocyclyl has 5 or 6 ring members and 1 to 2 heteroatoms selected from N, S, optionally annelated with phenyl; heterocyclyl-carbonyl(C₁₋₄)alkyl, wherein heterocyclyl has 5 or 6 ring members and 1 to 2 heteroatoms selected from N, S, optionally annelated with phenyl;
unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl.
R₂ is unsubstituted phenyl or phenyl substituted by (C₁₋₄)alkyl, (C₁₋₄)alkoxy or halogen,
A⁻ is as defined in claim 7.

13. The method of treatment of claim 7, wherein
R₁ is—unsubstituted (C₁₋₆)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms;
(C₆₋₁₈)aryl, (C₆₋₁₈)aryl(C₁₋₆)alkyl, (C₆₋₁₈)aryl-carbonyl(C₁₋₄)alkyl, heterocyclyl, heterocyclyl(C₁₋₆)alkyl, heterocyclyl-carbonyl(C₁₋₆)alkyl, wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₁₋₆)alkyl, (C₁₋₄)alkoxy, (C₃₋₈)cycloalkyl, nitro, halo(C₁₋₄)alkyl, halogen or sulfanyl(C₁₋₄)alkyl;
aminocarbonyl(C₁₋₆)alkyl in unsubstituted form or one or morefold substituted by
(C₁₋₆)alkyl;
(C₃₋₈)cycloalkyl,
halo(C₁₋₄)alkyl,
halogen,
unsubstituted (C₆₋₁₈)aryl,
(C₆₋₁₈)aryl substituted by (C₁₋₆)alkyl, (C₁₋₄)alkoxy, (C₃₋₈)cycloalkyl, nitro, halo(C₁₋₄)alkyl, halogen,
(C₆₋₁₈)aryl annelated with (C₆₋₁₈)aryl or heterocyclyl, wherein heterocyclyl has 5 or 6 ring members and 1 to 4 heteroatoms selected from N, O, S,
unsubstituted heterocyclyl, wherein heterocyclyl has 5 or 6 ring members and 1 to 4 heteroatoms selected from N, O, S,
heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₆₋₁₈)alkyl, halogen,
heterocyclyl annelated with (C₆₋₁₈)aryl or heterocyclyl, wherein heterocyclyl has 5 or 6 ring members and 1 to 4 heteroatoms selected from N, O, S,
R₂ is—unsubstituted (C₆₋₁₈)aryl or (C₆₋₁₈)aryl substituted by (C₁₋₄)alkyl, (C₁₋₄)alkoxy, (C₁₋₄)haloalkyl or halogen;
A⁻ is a pharmaceutically acceptable anion.

14. The method of treatment of claim 7, wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl,
unsubstituted phenyl or phenyl one or morefold substituted by (C₁₋₄)alkyl, (C₁₋₂)alkoxy, halogen, nitro;
unsubstituted phenyl(C₁₋₄)alkyl or phenyl(C₁₋₄)alkyl one or morefold substituted by (C₁₋₄)alkyl, (C₁₋₂)alkoxy, halogen, nitro;
unsubstituted phenyl-carbonyl(C₁₋₂)alkyl or phenyl-carbonyl(C₁₋₂)alkyl one or morefold substituted by (C₁₋₄)alkyl, (C₁₋₁₂)alkoxy, halogen, nitro, (C₃₋₈)cycloalkyl, (C₁₋₄)alkyl-sulfanyl,
heterocyclyl-(C₁₋₄)alkyl, wherein heterocyclyl has 5 or 6 ring members and 1 to 2 heteroatoms selected from N, S, optionally annelated with phenyl; heterocyclyl-carbonyl(C₁₋₄)alkyl, wherein heterocyclyl has 5 or 6 ring members and 1 to 2 heteroatoms selected from N, S, optionally annelated with phenyl;
unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl.
R₂ is unsubstituted phenyl or phenyl substituted by (C₁₋₄)alkyl, (C₁₋₄)alkoxy or halogen.

15. The method of treatment of claim 8 wherein the disease is an inflammatory or obstructive airways disease.

16. The pharmaceutical, composition of claim 10, wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl,
unsubstituted phenyl or phenyl one or morefold substituted by (C₁₋₄)alkyl, (C₁₋₂)alkoxy, halogen, nitro;
unsubstituted phenyl(C₁₋₄)alkyl or phenyl(C₁₋₄)alkyl one or morefold substituted by (C-4)alkyl, (C₁₋₂)alkoxy, nitro;
unsubstituted phenyl-carbonyl(C: 2)alkyl or phenyl-carbonyl(C₁₋₂)alkyl one or morefold substituted by (C₁₋₄)alkyl, (C₁₋₂)alkoxy, halogen, nitro, (C₃₋₈)cycloalkyl, (C₁₋₄)alkyl-sulfanyl,
heterocyclyl-(C₁₋₄)alkyl, wherein heterocyclyl has 5 or 6 ring members and i to 2 heteroatoms selected from N, S, optionally annelated with phenyl; heterocyclyl-carbonyl(C₁₋₄)alkyl, wherein heterocyclyl has 5 or 6 ring members and 1 to 2 heteroatoms selected from N, S, optionally annelated with phenyl;
unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl.
R₂ is unsubstituted phenyl or phenyl substituted by (C₁₋₄)alkyl, (C₁₋₄)alkoxy or halogen,
A⁻ is as defined in claim 10.

17. The pharmaceutical composition of claim 10, wherein
R₁ is—unsubstituted (C₁₋₈)alkyl or (C₁₋₆)alkyl one or morefold substituted by cyano, (C₁₋₄)alkyl-carbonyl, (C₁₋₄)alkoxy-carbonyl(C₁₋₂)alkyl-carbonyl, (C₃₋₈)cycloalkyl, nitro, halo(C₁₋₄)alkyl, halogen or heterocyclyl, wherein heterocyclyl has 5 to 6 ring members and 1 to 4 heteroatoms;
(C₆₋₁₈)aryl, (C₆₋₁₈)aryl(C₁₋₆)alkyl, (C₆₋₁₈)aryl-carbonyl(C₁₋₄)alkyl, heterocyclyl, heterocyclyl(C₁₋₆)alkyl, heterocyclyl-carbonyl(C₁₋₆)alkyl, wherein heterocyclyl has 5 or 6 ring members and 1 to 4 heteroatoms selected from N, O, S and wherein either (C₆₋₁₈)aryl or heterocyclyl or both are optionally annelated with (C₆₋₁₈)aryl or heterocyclyl, in unsubstituted form or one or morefold substituted by (C₆₋₁₈)alkyl, (C₁₋₄)alkoxy,
(C₃₋₈)cycloalkyl, nitro, halo(C₁₋₄)alkyl, halogen or sulfanyl(C₁₋₄)alkyl;
aminocarbonyl(C₁₋₆)alkyl in unsubstituted form or one or morefold substituted by
(C₁₋₆)alkyl;
(C₃₋₈)cycloalkyl,
halo(C₁₋₄)alkyl,
halogen,
unsubstituted (C₆₋₁₈)aryl,
(C₆₋₁₈)aryl substituted by (C₁₋₆)alkyl, (C₁₋₄)alkoxy, (C₃₋₈)cycloalkyl, nitro, halo(C₁₋₄)alkyl, halogen,
(C₆₋₁₈)aryl annelated with (C₆₋₁₈)aryl or heterocyclyl, wherein heterocyclyl has 5 or 6 ring members and 1 to 4 heteroatoms selected from N, O, S,
unsubstituted heterocyclyl, wherein heterocyclyl has 5 or 6 ring members and 1 to 4 heteroatoms selected from N, O, S,
heterocyclyl, wherein heterocyclyl has 5 or 6 ring members and 1 to 4 heteroatoms selected from N, O, S, substituted by unsubstituted (C₆₋₁₈)aryl or (C₆₋₁₈)aryl substituted by (C₁₋₆)alkyl, (C₁₋₄)alkoxy, (C₃₋₈)cycloalkyl, nitro, halo(C₁₋₄)alkyl, halogen,
heterocyclyl annelated with (C₆₋₁₈)aryl or heterocyclyl, wherein heterocyclyl has 5 or 6 ring members and 1 to 4 heteroatoms selected from N, O, S,
R₂ is—unsubstituted (C₆₋₁₈)aryl or (C₆₋₁₈)aryl substituted by (C₁₋₄)alkyl, (C₁₋₄)alkoxy, (C₁₋₄)haloalkyl or halogen;
A⁻ is a pharmaceutically acceptable anion.

18. The pharmaceutical composition of claim 10, wherein
R₁ is (C₁₋₄)alkyl, cyano(C₁₋₄)alkyl, (C₁₋₂)alkyl-carbonyl(C₁₋₂)alkyl, (C₁₋₄)alkoxy-carbonyl-(C₁₋₂)alkyl-carbonyl(C₁₋₂)alkyl,
unsubstituted phenyl or phenyl one or morefold substituted by (C₁₋₄)alkyl, (C₁₋₂)alkoxy, halogen, nitro;
unsubstituted phenyl(C₁₋₄)alkyl or phenyl(C₁₋₄)alkyl one or morefold substituted by (C₁₋₄)alkyl, (C₁₋₂)alkoxy, halogen, nitro;
unsubstituted phenyl-carbonyl(C₁₋₂)alkyl or phenyl-carbonyl(C₁₋₂)alkyl one or morefold substituted by (C₁₋₄)alkyl, (C₁₋₂)alkoxy, halogen, nitro, (C₃₋₈)cycloalkyl, (C₁₋₄)alkyl-sulfanyl,
heterocyclyl-(C₁₋₄)alkyl, wherein heterocyclyl has 5 or 6 ring members and 1 to 2 heteroatoms selected from N, S, optionally annelated with phenyl; heterocyclyl-carbonyl(C₁₋₄)alkyl, wherein heterocyclyl has 5 or 6 ring members and 1 to 2 heteroatoms selected from N, S, optionally annelated with phenyl;
unsubstituted amino-carbonyl(C₁₋₂)alkyl or amino-carbonyl(C₁₋₂)alkyl one or morefold substituted by (C₁₋₄)alkyl, unsubstituted or substituted (C₆₋₁₈)aryl, unsubstituted or substituted phenyl(C₁₋₁₂)alkyl, (C₃₋₆)cycloalkyl, unsubstituted or substituted heterocyclyl, wherein heterocyclyl has 5 or 6 ring members and 1 to 2 heteroatoms selected from N, S, optionally annelated with phenyl.
R₂ is unsubstituted phenyl or phenyl substituted by (C₁₋₄)alkyl, (C₁₋₄)alkoxy or halogen.
